(19) United States
(12) Patent Application Publication
(10) Pub. No.: US 2008/0275315 A1
(43) Pub. Date: Nov. 6, 2008

(54) Pigmentary Deposition Portion Remote Diagnosis System

(76) Inventors: Hiroshi Oka, Tokyo (JP); Masaru Tanaka, Tokyo (JP); Toshiyuki Tanaka, Kanagawa (JP); Hitoshi Iyatomi, Kanagawa (JP); Masafumi Hagiwara, Kanagawa (JP); Masao Yamamoto, Tokyo (JP)

Correspondence Address: PAUL, HASTINGS, JANOFSKY & WALKER LLP, 875 15th Street, NW, Washington, DC, 20005, US

(21) Appl. No.: 10/585,457

(22) PCT Filed: Jan. 11, 2005
(86) PCT No.: PCT/JP2005/000195

§ 371 Date: May 7, 2008

(30) Foreign Application Priority Data
Jan. 9, 2004 (JP) 2004-004747

(51) Int. Cl. A61B 5/00; G06Q 50/00

(52) U.S. Cl. 600/306; 705/3

(57) ABSTRACT

A major object of this invention is to provide a remote diagnosis apparatus, a remote diagnosis system, a user terminal, a program, a diagnosis program, and a storage for easily diagnosing a pigmentary deposition portion. A user of the system picks up the skin having benign nevus pigmentosus that might be melanoma through a dermoscope using a portable telephone with a camera to which the dermoscope is attached by means of an adapter, accesses to the Internet using an Internet connection function of the portable telephone, and sends the picked up skin image to the remote diagnosis apparatus. After receiving the skin image, the remote diagnosis apparatus uses the melanoma diagnosis program to examine the skin image for melanoma and a disease stage of melanoma if there is melanoma and then sends a result to the user.

### TECHNICAL FIELD

This invention relates to a remote diagnosis apparatus to be used for performing a remote diagnosis of a pigmentary deposition portion, a remote diagnosis system, a user terminal, a program, a diagnosis program, and a storage.

### BACKGROUND ART

Melanocyte (pigmentary cell) exists all over the basal lamina of the epidermis. When the melanocyte grows to be melanoma (malignant melanoma), abnormal proliferation of the cell occurs to spread from the basal lamina to the dermis which is under the basal lamina. When the melanoma spreads to a certain degree (less than 1 mm to a several millimeters), the tumor cells spread further in the vertical direction to form an ulcer (a hole is formed due to destruction of cell surfaces to allow the lower lamina to be seen). After that, the tumor cells develop metastasis to spread to the adjacent lymphatic node.

In the case where the melanoma is diagnosed in its early stage, particularly when the diagnosis is made when the melanoma has a thickness of 0.75 mm or less, such melanoma is not life-threatening and results in good prognosis (5-year survival rate is 93% or more; see, for example, Cancer, vol. 62, p 1207-1214, USA, 1988).

### DISCLOSURE OF THE INVENTION

### Problems to be Solved by the Invention Though the melanoma is lower in occurrence frequency as compared to other types of tumors, it has a high malignancy and is rapidly progressive to invade and spread in a shorter period of time as compared to other types of skin cancers such as basal cell cancer and squamous cell cancer. Also, there are few effective therapeutic methods for the case of a lesion in which the tumor cells generate distant metastasis. Further, it is difficult to distinguish the melanoma from benign nevus pigmentosus with the naked eye, so that the progression cannot be determined in many cases. For the above reasons, patients with the melanoma tend to take long to make their first visit to hospitals, and the melanoma is found at the advanced stage which is difficult to cure.

Therefore, a major object of this invention is to provide a remote diagnosis apparatus, a remote diagnosis system, a user terminal, a program, a diagnosis program, and a storage to be used for conveniently diagnosing a remote pigmentary deposition portion.

### Means for Solving the Problems

A remote diagnosis apparatus according to this invention is a remote diagnosis apparatus for diagnosing a skin lesion, which is communicably connected to a first user terminal and a second user terminal provided with a camera device with dermoscope, comprising: a database for storing data which are received from the first user terminal and relate to the skin lesion, and a diagnosis program updated based on the data stored in the database and diagnosing skin images for the skin lesion, the remote diagnosis apparatus receiving a skin image of a pigmentary deposition portion picked up by the camera device with dermoscope from the second user terminal; diagnosing the received skin image for the skin lesion with the use of the diagnosis program; and sending a diagnosis result to a predetermined destination.

As used herein, the pigmentary deposition portion means a pigmented region of a skin. The pigmentary deposition may be that caused by a skin lesion such as melanoma which is a subject of treatment or that having no skin lesion such as pigmented macule which is not the subject of treatment.

The remote diagnosis apparatus according to this invention may be a remote diagnosis apparatus communicably connected to a first user terminal and a second user terminal provided with a camera device with dermoscope, comprising: a database for storing data which are received from the first user terminal and relate to a skin lesion; a diagnosis program which is updated based on the data stored in the database and diagnoses skin images for the skin lesion; and an image storage for storing the skin images to be diagnosed, the remote diagnosis apparatus receiving a skin image of a pigmentary deposition portion picked up by the camera device with dermoscope and source information for identifying the pigmentary deposition portion from the second user terminal and storing the received skin image in the image storage after associating the source information with the skin image, and the diagnosis program taking out the skin image stored in the image storage to diagnose the skin image for the skin lesion and sending a diagnosis result to a destination designated by the source information.

The remote diagnosis apparatus according to this invention may be a remote diagnosis apparatus communicably connected to a first user terminal and a second user terminal provided with a camera device with dermoscope, comprising: a data receiving unit for receiving data relating to a skin lesion from the first user terminal; a database for storing data relating to the skin lesion, which stores the received data; a receiving unit for receiving a skin image of a pigmentary deposition portion picked up by the camera device with dermoscope and source information for identifying the pigmentary deposition portion from the second user terminal; an image storage for storing the received skin image after associating the source information with the skin image; a diagnosis program which is updated based on the data stored in the database and takes out the skin image stored in the image storage to diagnose the skin image for the skin lesion; and a sending unit for sending a diagnosis result of the diagnosis program to a destination designated by the source information.

The remote diagnosis apparatus according to this invention may be a remote diagnosis apparatus communicably connected to a first user terminal and a second user terminal provided with a camera device with dermoscope, comprising: a database for storing data which are received from the first user terminal and relate to a skin lesion; a first diagnosis program which is updated based on the data stored in the database and diagnoses skin images for the skin lesion; and a second diagnosis program for diagnosing based on a comparison of plural diagnosis results for the skin lesion, the remote diagnosis apparatus receiving a first skin image of a pigmentary deposition portion picked up by the camera device with dermoscope from the second user terminal to diagnose the first skin image for the skin lesion with the use of the first diagnosis program, storing a diagnosis result for the first skin image, receiving a second skin image of the pigmentary deposition portion same as the first skin image picked up by the camera device with dermoscope at a different time point from the second user terminal and diagnosing the second skin image for the skin lesion with the use of the first diagnosis program, diagnosing the skin lesion by comparing the diagnosis result for the first skin image with a diagnosis result for the second skin image with the use of the second diagnosis program, and sending at least one of the diagnosis results of the first diagnosis program and the second diagnosis program to a predetermined destination.

The remote diagnosis apparatus according to this invention may be a remote diagnosis apparatus communicably connected to a first user terminal and a second user terminal provided with a camera device with dermoscope, comprising: a database for storing data which are received from the first user terminal and relate to a skin lesion; a first diagnosis program which is updated based on the data stored in the database and diagnoses skin images for the skin lesion; a second diagnosis program for diagnosing based on a comparison of plural diagnosis results for the skin lesion; an image storage for storing the skin images to be diagnosed; and a diagnosis result storage for storing a diagnosis result, the remote diagnosis apparatus receiving a first skin image of a pigmentary deposition portion picked up by the camera device with dermoscope, source information for identifying the pigmentary deposition portion, and first time information relating to a time of sending the first skin image from the second user terminal, storing the first skin image in the image storage after associating the source information and the first time information with the first skin image, taking out the first skin image stored in the image storage to diagnose the first skin image for the skin lesion with the use of the first diagnosis program, storing a diagnosis result for the first skin image in the diagnosis result storage after associating the source information and the first time information with the diagnosis result, receiving a second skin image of the pigmentary deposition portion same as the first skin image picked up by the camera device with dermoscope at a different time point, source information for identifying the pigmentary deposition portion, and second time information relating to a time of sending the second skin image from the second user terminal, storing the second skin image in the image storage after associating the source information and the second time information with the second skin image, taking out the second skin image stored in the image storage to diagnose the second skin image for the skin lesion with the use of the first diagnosis program, comparing the diagnosis result for the first skin image with a diagnosis result for the second skin image with the use of the second diagnosis program to diagnose further for the skin lesion, and sending at least one of the diagnosis results of the first diagnosis program and the second diagnosis program to a destination designated by the source information.

The remote diagnosis apparatus according to this invention may be a remote diagnosis apparatus communicably connected to a first user terminal and a second user terminal provided with a camera device with dermoscope, comprising: a database for storing data relating to a skin lesion; a data receiving unit for receiving data relating to the skin lesion from the first user terminal; a database for storing data relating to the skin lesion, which stores the received data; a receiving unit for receiving a skin image of a pigmentary deposition portion picked up by the camera device with dermoscope, source information for identifying the pigmentary deposition portion, and time information relating to a time of sending the skin image from the second user terminal; an image storage for storing the received skin image after associating the source information and the time information with the skin image; a first diagnosis program which is updated based on the data stored in the database and takes out the skin image stored in the image storage to diagnose the skin image for the skin lesion; a diagnosis result storage for storing a diagnosis result after associating the source information and the time information with the diagnosis result; a second diagnosis program capable of diagnosing the skin lesion based on plural diagnosis results for the skin lesion having different time information; and a sending unit for sending at least one of diagnosis results of the first diagnosis program and the second diagnosis program to a destination designated by the source information.

Note that each of the second user terminals may preferably be provided with a portable telephone function. Also, each of the second user terminals may preferably communicate via the Internet.

The remote diagnosis apparatus according to this invention may be a remote diagnosis apparatus communicably connected to a database control server provided with a database for storing data relating to a skin lesion and a user terminal provided with a camera device with dermoscope, comprising a diagnosis program which is updated based on the data stored in the database and diagnoses skin images for the skin lesion, the remote diagnosis apparatus receiving a skin image of a pigmentary deposition portion picked up by the camera device with dermoscope from the user terminal, diagnosing the received skin image for the skin lesion with the use of the diagnosis program, and sending a diagnosis result to a predetermined destination.

The remote diagnosis apparatus according to this invention may be a remote diagnosis apparatus communicably connected to a database control server provided with a database for storing data relating to a skin lesion and a user terminal provided with a camera device with dermoscope, comprising a diagnosis program which is updated based on the data stored in the database and diagnoses skin images for the skin lesion and an image storage for storing the skin images to be diagnosed, the remote diagnosis apparatus receiving a skin image of a pigmentary deposition portion picked up by the camera device with dermoscope and source information for identifying the pigmentary deposition portion from the user terminal, storing the received skin image in the image storage after associating the source information with the skin image, diagnosing the skin image stored in the image storage for the skin lesion with the use of the diagnosis program, and sending a diagnosis result to a destination designated by the source information.

The remote diagnosis apparatus according to this invention may be a remote diagnosis apparatus communicably connected to a database control server provided with a database for storing data relating to a skin lesion and a user terminal provided with a camera device with dermoscope, comprising: a receiving unit for receiving a skin image of a pigmentary deposition portion picked up by the camera device with dermoscope and source information for identifying the pigmentary deposition portion from the user terminal; an image storage for storing the received skin image after associating the source information with the skin image; a diagnosis program which is updated based on the data stored in the database and takes out the skin image stored in the image storage to diagnose the skin image for the skin lesion; and a sending unit for sending a diagnosis result of the diagnosis program to a destination designated by the source information.

The remote diagnosis apparatus according to this invention may be a remote diagnosis apparatus communicably connected to a database control server provided with a database for storing data relating to a skin lesion and a user terminal provided with a camera device with dermoscope, comprising a first diagnosis program which is updated based on the data stored in the database and diagnoses skin images for the skin lesion and a second diagnosis program for diagnosing based on plural diagnosis results for the skin lesion, the remote diagnosis apparatus receiving a first skin image of a pigmentary deposition portion picked up by the camera device with dermoscope from the user terminal, diagnosing the first skin image for the skin lesion with the use of the first diagnosis program, storing a diagnosis result for the first skin image, receiving a second skin image of the pigmentary deposition portion same as the first skin image picked up by the camera device with dermoscope at a different time point from the user terminal, diagnosing the second skin image for the skin lesion with the use of the first diagnosis program, comparing the diagnosis result for the first skin image with a diagnosis result for the second skin image with the use of the second diagnosis program, and sending at least one of the diagnosis results of the first diagnosis program and the second diagnosis program to a destination designated by the source information.

The remote diagnosis apparatus according to this invention may be a remote diagnosis apparatus communicably connected to a database control server provided with a database for storing data relating to a skin lesion and a user terminal provided with a camera device with dermoscope, comprising: a first diagnosis program which is updated based on the data stored in the database and diagnoses skin images for the skin lesion; a second diagnosis program for diagnosing based on plural diagnosis results for the skin lesion; an image storage for storing the skin images to be diagnosed; and a diagnosis result storage for storing a diagnosis result, the remote diagnosis apparatus receiving a first skin image of a pigmentary deposition portion picked up by the camera device with dermoscope, source information for identifying the pigmentary deposition portion, and first time information relating to a time of sending the first skin image from the user terminal, storing the first skin image in the image storage after associating the source information and the first time information with the first skin image, taking out the first skin image stored in the image storage to diagnose the first skin image for the skin lesion with the use of the first diagnosis program, storing a diagnosis result for the first skin image in the diagnosis result storage after associating the source information and the first time information with the diagnosis result, receiving a second skin image of the pigmentary deposition portion same as the first skin image picked up by the camera device with dermoscope at a different time point, source information for identifying the pigmentary deposition portion, and second time information relating to a time of sending the second skin image from the user terminal, storing the second skin image in the diagnosis result storage after associating the source information with the second skin image, taking out the stored second skin image to diagnose the second skin image for the skin lesion with the use of the first diagnosis program, diagnosing through a comparison between the diagnosis result for the first skin image and a diagnosis result for the second skin image with the use of the second diagnosis program, and sending at least one of the diagnosis results of the first diagnosis program and the second diagnosis program to a destination designated by the source information.

The remote diagnosis apparatus according to this invention may be a remote diagnosis apparatus communicably connected to a database control server provided with a database for storing data relating to a skin lesion and a user terminal provided with a camera device with dermoscope, comprising: a receiving unit for receiving a skin image of a pigmentary deposition portion picked up by the camera device with dermoscope, source information for identifying the pigmentary deposition portion, and time information relating to a time of sending the skin image from the user terminal; an image storage for storing the received skin image after associating the source information and the time information with the skin image; a first diagnosis program which is updated based on the data stored in the database and takes out the skin image stored in the image storage to diagnose the skin image for a skin lesion; a diagnosis result storage for storing a diagnosis result for the skin image after associating the source information and the time information with the diagnosis result; a second diagnosis program capable of diagnosing the skin lesion based on plural diagnosis results for the skin lesion of the skin image, which are stored in the diagnosis result storage and different in time information; and a sending unit for sending at least one of diagnosis results of the first diagnosis program and the second diagnosis program to a destination designated by the source information.

The remote diagnosis apparatus according to this invention may be a remote diagnosis apparatus communicably connected to a user terminal provided with a camera device with dermoscope, comprising receiving a skin image of a pigmentary deposition portion picked up by the camera device with dermoscope; diagnosing the received skin image for the skin lesion; and sending a diagnosis result to a predetermined destination.

The remote diagnosis apparatus according to this invention may be a remote diagnosis apparatus communicably connected to a user terminal provided with a camera device with dermoscope, comprising a diagnosis program for diagnosing skin images for the skin lesion and an image storage for storing the skin images to be diagnosed; the remote diagnosis apparatus receiving a skin image of a pigmentary deposition portion picked up by the camera device with dermoscope and source information for identifying the pigmentary deposition portion, storing the received skin image in the image storage after associating the source information with the skin image, taking out the skin image stored in the image storage and diagnosing the skin image for the skin lesion with the use of the diagnosis program, and sending a diagnosis result to a destination designated by the source information.

The remote diagnosis apparatus according to this invention may be a remote diagnosis apparatus communicably connected to a user terminal provided with a camera device with dermoscope, comprising: a receiving unit for receiving a skin image of a pigmentary deposition portion picked up by the camera device with dermoscope and source information for identifying the pigmentary deposition portion; an image storage for storing the received skin image after associating the source information with the skin image; a diagnosis program for taking out the skin image stored in the image storage to diagnose the skin image for the skin lesion; and a sending unit for sending a diagnosis result of the diagnosis program to a destination designated by the source information.

Note that each of the user terminals may preferably be provided with a portable telephone function. Also, each of the second terminals may preferably communicate via the Internet.

Also, each of the dermoscopes may preferably be provided with a polarizing filter. Also, the skin lesion which is the diagnosis subject may preferably be melanoma.

The remote diagnosis system according to this invention may be a remote diagnosis system for diagnosing a skin lesion, comprising: a first user terminal; a second user terminal provided with a camera device with dermoscope; a remote diagnosis apparatus communicably connected to the first user terminal and the second user terminal and comprises a database for storing data which are received from the first user terminal and relate to the skin lesion; and a diagnosis program relating to the skin lesion which is updated based on the data stored in the database, wherein the second user terminal sends a skin image of a pigmentary deposition portion picked up by the camera device with dermoscope to the remote diagnosis apparatus, and the remote diagnosis apparatus receives the skin image, diagnoses the received skin image for the skin lesion with the use of the diagnosis program, and sends a diagnosis result to a predetermined destination.

The remote diagnosis system according to this invention may be a remote diagnosis system for diagnosing a skin lesion, comprising: a first user terminal; a second user terminal provided with a camera device with dermoscope; and a remote diagnosis apparatus communicably connected to the first and the second user terminals, wherein the first user terminal comprises a sending unit for sending data relating to the skin lesion to the remote diagnosis apparatus, the second user terminal comprises a sending unit for sending a skin image of a pigmentary deposition portion picked up by the camera device with dermoscope to the remote diagnosis apparatus, and the remote diagnosis apparatus comprises a data receiving unit for receiving the data from the first user terminal, a database for storing the data which are received from the first user terminal and relate to the skin lesion, an image receiving unit for receiving the skin image from the second user terminal, a diagnosis program which is updated based on the data stored in the database and diagnoses the skin image received from the second user terminal for the skin lesion, and a sending unit for sending a diagnosis result to a predetermined destination.

The remote diagnosis system according to this invention may be a remote diagnosis system for diagnosing a skin lesion, comprising: a first user terminal; a database control server communicably connected to the first user terminal and provided with a database for storing data which are received from the first user terminal and relate to the skin lesion; a second user terminal provided with a camera device with dermoscope; and a remote diagnosis apparatus communicably connected to the database control server and the second user terminal and provided with a diagnosis program which is updated based on the data stored in the database and diagnoses skin images for the skin lesion, wherein the second user terminal sends a skin image of a pigmentary deposition portion picked up by the camera device with dermoscope; and the remote diagnosis apparatus diagnoses the skin image received from the second user terminal for the skin lesion with the use of the diagnosis program and sends a diagnosis result to a predetermined destination.

The remote diagnosis system according to this invention may be a remote diagnosis system for diagnosing a skin lesion, comprising: a first user terminal; a database control server communicably connected to the first user terminal; a second user terminal provided with a camera device with dermoscope; and a remote diagnosis apparatus communicably connected to the database control server and the second user terminal, wherein the first user terminal comprises a sending unit for sending data relating to the skin lesion to the remote diagnosis apparatus, the database control server comprises a data receiving unit for receiving the data from the first user terminal and a database for storing the data which are received from the first user terminal and relate to the skin lesion; the second user terminal comprises a sending unit for sending a skin image of a pigmentary deposition portion picked up by the camera device with dermoscope to the remote diagnosis apparatus, and the remote diagnosis apparatus comprises a receiving unit for receiving the skin image from the second user terminal; a diagnosis program which is updated based on the data stored in the database and diagnoses the skin image received from the second user terminal for the skin lesion; and a sending unit for sending a diagnosis result to a predetermined destination.

The remote diagnosis system according to this invention may be a remote diagnosis system for diagnosing a skin lesion, comprising: a first user terminal; a second user terminal provided with a camera device with dermoscope; and a remote diagnosis apparatus communicably connected to the first and the second user terminals and comprises a database for storing data which are received from the first user terminal and relate to the skin lesion, a first diagnosis program which is updated based on the data stored in the database and diagnoses skin images for the skin lesion, and a second diagnosis program for diagnosing based on plural diagnosis results for the skin lesion, wherein the second user terminal sends a first skin image of a pigmentary deposition portion picked up by the camera device with dermoscope to the remote diagnosis apparatus; the remote diagnosis apparatus receives the first skin image and diagnoses the received skin image for the skin lesion with the use of the first diagnosis program; the second user terminal sends a second skin image of the pigmentary deposition portion same as the first skin image picked up by the camera device with dermoscope at a different time point to the remote diagnosis apparatus; the remote diagnosis apparatus receives the second skin image and diagnoses the received second skin image for the skin lesion with the use of the first diagnosis program; the remote diagnosis apparatus diagnoses the skin lesion by comparing the diagnosis result for the first skin image with a diagnosis result for the second skin image with the use of the second diagnosis program and sends at least one of the diagnosis results of the first diagnosis program and the second diagnosis program to a predetermined destination.

The remote diagnosis system according to this invention may be a remote diagnosis system for diagnosing a skin lesion, comprising: a first user terminal; a second user terminal provided with a camera device with dermoscope; and a remote diagnosis apparatus communicably connected to the first and the second user terminals, wherein the first user terminal comprises a sending unit for sending data relating to the skin lesion to the remote diagnosis apparatus; the second user terminal comprises a sending unit for sending a skin image of a pigmentary deposition portion picked up by the camera device with dermoscope, source information for identifying the pigmentary deposition portion, and time information relating to a time of sending the skin image to the remote diagnosis apparatus; and the remote diagnosis apparatus comprises a data receiving unit for receiving the data from the first user terminal; a database for storing the data which are received from the first user terminal and relate to the skin lesion; an image receiving unit for receiving the skin image, the source information, and the time information from the second user terminal; an image storage for storing the received skin image after associating the source information and the time information with the skin image; a first diagnosis program which is updated based on the data stored in the database and diagnoses the skin image taken out from the storage for the skin lesion; a second diagnosis program capable of diagnosing the skin lesion based on plural diagnosis results for the skin lesion of the skin image, which are stored in the diagnosis result storage and different in time information, the diagnosis results being stored in the diagnosis result storage; and a sending unit for sending at least one of diagnosis results of the first diagnosis program and the second diagnosis program to a predetermined destination.

The remote diagnosis system according to this invention may be a remote diagnosis system for diagnosing a skin lesion, comprising: a first user terminal; a database control server communicably connected to the first user terminal and provided with a database for storing data which are received from the first user terminal and relate to the skin lesion; a second user terminal provided with a camera device with dermoscope; and a remote diagnosis apparatus communicably connected to the second user terminal and comprising a first diagnosis program which is updated based on the data stored in the database and diagnoses skin images for the skin lesion, and a second diagnosis program for diagnosing the skin lesion based on a comparison of plural diagnosis results, wherein the second user terminal sends a first skin image of a pigmentary deposition portion picked up by the camera device with dermoscope to the remote diagnosis apparatus; the remote diagnosis apparatus receives the first skin image and diagnoses the received first skin image for the skin lesion with the use of the first diagnosis program; the second user terminal sends a second skin image of the pigmentary deposition portion same as the first skin image picked up by the camera device with dermoscope to the remote diagnosis apparatus; the remote diagnosis apparatus receives the second skin image, diagnoses the received second skin image for the skin lesion with the use of the first diagnosis program, diagnoses the skin lesion based on a comparison between the diagnosis result for the first skin image and a diagnosis result for the second skin image with the use of the second diagnosis program, and sends at least one of the diagnosis results of the first diagnosis program and the second diagnosis program to the second user terminal.

The remote diagnosis system according to this invention may be a remote diagnosis system for diagnosing a pigmentary deposition portion, comprising: a first user terminal; a database control server communicably connected to the first user terminal; a second user terminal provided with a camera device with dermoscope; and a remote diagnosis apparatus communicably connected to the database control server and the second user terminal, wherein the first user terminal comprises a sending unit for sending data relating to the skin lesion to the remote diagnosis apparatus; the database control server comprises a data receiving unit for receiving the data from the first user terminal and a database for storing the data received from the first user terminal; the second user terminal comprises a sending unit for sending a skin image of a pigmentary deposition portion picked up by the camera device with dermoscope, source information for identifying the pigmentary deposition portion, and time information relating to a time of sending the skin image to the remote diagnosis apparatus; and the remote diagnosis apparatus comprises an image receiving unit for receiving the skin image, the source information, and the time information from the second user terminal; an image storage for storing the skin image after associating the source information and the time information with the skin image; a first diagnosis program which is updated based on the data stored in the database and diagnoses the received skin image for the skin lesion; a second diagnosis program capable of diagnosing based on plural diagnosis results for the skin lesion; a diagnosis result storage for storing the diagnosis results; and a sending unit for sending the diagnosis result to a predetermined destination.

Note that each of the second user terminals may preferably be provided with a portable telephone function. Also, each of the second user terminals may preferably communicate via the Internet.

The remote diagnosis system according to this invention may be a remote medial system for diagnosing a skin lesion, comprising: a user terminal provided with a camera device with dermoscope and a remote diagnosis apparatus communicably connected to the user terminal, wherein the user terminal sends a skin image of a pigmentary deposition portion picked up by the camera device with dermoscope and source information for identifying the pigmentary deposition portion to the remote diagnosis apparatus, and the remote diagnosis apparatus diagnoses the skin image received from the user terminal for the skin lesion to send a diagnosis result to a destination designated by the source information.

The remote diagnosis system according to this invention may be a remote medical diagnosis system for diagnosing presence/absence and a disease state of a skin lesion, comprising: a user terminal provided with a camera device with dermoscope and a remote diagnosis apparatus communicably connected to the user terminal, wherein the user terminal comprises a sending unit for sending a skin image of a pigmentary deposition portion picked up by the camera device with dermoscope to the remote diagnosis apparatus, and the remote diagnosis apparatus comprises a receiving unit for receiving the skin image of the pigmentary deposition portion picked up by the camera device with dermoscope and source information for identifying the pigmentary deposition portion; an image storage for storing the received skin image after associating the source information with the skin image; a diagnosis program taking out the skin image stored in the image storage to diagnose the skin image for the skin lesion; and a sending unit for sending a diagnosis result obtained by the diagnosis to a destination designated by the source information.

The remote diagnosis system according to this invention may be a remote diagnosis system for diagnosing a skin lesion, comprising: a user terminal provided with a camera device with dermoscope; a remote diagnosis apparatus communicably connected to the user terminal; a first diagnosis program diagnosing skin images for the skin lesion; and a second diagnosis program for diagnosing based on plural diagnosis results for the skin lesion, wherein the user terminal sends a first skin image of a pigmentary deposition portion picked up by the camera device with dermoscope to the remote diagnosis apparatus; the remote diagnosis apparatus receives the first skin image and diagnoses the received first skin image for the skin lesion with the use of the first diagnosis program, and the user terminal sends a second skin image of the pigmentary deposition portion same as the first skin image picked up by the camera device with dermoscope to the remote diagnosis apparatus, and the remote diagnosis apparatus receives the second skin image, diagnoses the second skin image for the skin lesion with the use of the first diagnosis program, diagnoses the skin lesion based on a comparison between the diagnosis result for the first skin image and a diagnosis result for the second skin image with the use of the second diagnosis program, and sends at least one of the diagnosis results of the first diagnosis program and the second diagnosis program to a destination designated by the source information.

The remote diagnosis system according to this invention may be a remote medical diagnosis system for diagnosing presence/absence and a disease state of a skin lesion and may comprise: a user terminal provided with a camera device with dermoscope and a remote diagnosis apparatus communicably connected to the user terminal, wherein the remote diagnosis apparatus comprises: an image receiving unit for receiving a skin image of a pigmentary deposition portion picked up by the camera device with dermoscope, source information for identifying the pigmentary deposition portion, and time information relating to a time of sending the skin image; an image storage for storing the received skin image after associating the source information and the time information with the skin image; a first diagnosis program for taking out the skin image from the image storage to diagnose the skin image for the skin lesion; a diagnosis result storage for storing a diagnosis result after associating the source information and the time information with the diagnosis result; a second diagnosis program for diagnosing the skin lesion based on plural diagnosis results for the skin lesion having different time information; and a sending unit for sending at least one of diagnosis results of the first diagnosis program and the second diagnosis program to a destination designated by the source information.

Note that each of the user terminals in the above-described systems may preferably be provided with a portable telephone function. Also, each of the user terminals may preferably communicate via the Internet. Also, each of the dermoscopes may preferably be provided with a polarizing filter. Also, the skin lesion which is a subject of the diagnosis may preferably be melanoma.

A user terminal according to this invention comprises a camera device with dermoscope provided with a polarizing filter; a sending unit capable of sending a skin image of a pigmentary deposition portion picked up by the camera device with dermoscope and source information for identifying the pigmentary deposition portion; a receiving unit capable of receiving a diagnosis result obtained by diagnosing the skin image for a skin lesion; and a portable telephone function.

In the user terminal, it is preferred that the sending/receiving unit may send/receive to/from any one of the remote diagnosis apparatuses. The sending unit may further send time information relating to a time of sending. It is preferable to communicate with the remote diagnosis apparatus via the Internet. The skin lesion which is a subject of the diagnosis may preferably be melanoma.

A program according to this invention is a program for causing a computer to execute a remote diagnosis for diagnosing a skin lesion, the computer being communicably connected to a first user terminal and a second user terminal provided with a camera device with dermoscope and comprising a database for storing data which are received from the first user terminal and relate to the skin lesion, a diagnosis program which is updated based on the data stored in the database and diagnoses skin images for the skin lesion, and an image storage for storing the skin images to be diagnosed, wherein the program causes the computer to execute a step for receiving a skin image of a pigmentary deposition portion picked up by the camera device with dermoscope and source information for identifying the pigmentary deposition portion from the second user terminal; a step for storing the received skin image in the image storage after associating the source information with the skin image; a step for taking out the skin image stored in the image storage and diagnosing the skin image for the skin lesion; and a step for sending a diagnosis result to a destination designated by the source information.

The program according to this invention is a program for causing a computer to execute a remote diagnosis for diagnosing presence/absence and a disease state of a skin lesion, the computer being communicably connected to a first user terminal and a second user terminal provided with a camera device with dermoscope and comprising a database for storing data which are received from the first user terminal and relate to the skin lesion, a first diagnosis program which is updated based on the data stored in the database and diagnoses skin images for the skin lesion, a second diagnosis program for diagnosing the skin lesion based on plural diagnosis results for the skin lesion, an image storage for storing the skin images to be diagnosed, and a diagnosis result storage for storing a diagnosis result, wherein the program may cause the computer to execute a step for receiving a first skin image of a skin lesion picked up by the camera device with dermoscope, source information for identifying the skin lesion, and time information relating to a time of sending the skin image from the second user terminal, a step for storing the first skin image in the image storage after associating the source information and the time information with the first skin image; a step for taking out the first skin image stored in the image storage to diagnose the first skin image for the skin lesion with the use of the first diagnosis program; a step for storing a diagnosis result for the first skin image in the diagnosis result storage after associating the source information and the time information with the diagnosis result; a step for receiving a second skin image of the pigmentary deposition portion same as the first skin image picked up by the camera device with dermoscope at a different time point, source information for identifying the pigmentary deposition portion, and time information relating to a time of sending the skin image from the second user terminal, a step for storing the second skin image in the image storage after associating the source information and the time information with the second skin image; a step for taking out the second skin image stored in the image storage to diagnose the second skin image for the skin lesion with the use of the first diagnosis program; a step for diagnosing the skin lesion based on a comparison between the diagnosis result for the first skin image and a diagnosis result for the second skin image with the use of the second diagnosis program; and a step for sending at least one of the diagnosis results of the first diagnosis program and the second diagnosis program to a destination designated by the source information.

The program according to this invention is a program for causing a computer to execute a remote diagnosis for diagnosing a skin lesion, the computer being communicably connected to a database control server communicably connected to the first user terminal and provided with a database for storing data which are received from the first user terminal and relate to the skin lesion and a second user terminal provided with a camera device with dermoscope and comprising a diagnosis program which is updated based on the data stored in the database and diagnoses skin images for the skin lesion and an image storage for storing the skin images to be diagnosed, wherein the program may cause the computer to execute a step for receiving a skin image of a pigmentary deposition portion picked up by the camera device with dermoscope and source information for identifying the pigmentary deposition portion from the second user terminal; a step for storing the received skin image in the image storage after associating the source information with the skin image; a step for taking out the stored skin image and diagnosing the skin image for the skin lesion with the use of the diagnosis program; and a step for sending a diagnosis result to a destination designated by the source information.

The program according to this invention is a program for causing a computer to execute a remote diagnosis for diagnosing a skin lesion, the computer being communicably connected to a database control server communicably connected to the first user terminal and provided with a database for storing data which are received from the first user terminal and relate to the skin lesion and a second user terminal provided with a camera device with dermoscope and comprising a first diagnosis program which is updated based on the data stored in the database and diagnoses skin images for the skin lesion; a second diagnosis program for diagnosing the skin lesion based on plural diagnosis results for the skin lesion; an image storage for storing the skin images to be diagnosed; and a diagnosis result storage for storing a diagnosis result, wherein the program may cause the computer to execute a step for receiving a first skin image of a pigmentary deposition portion picked up by the camera device with dermoscope, source information for identifying the pigmentary deposition portion, and time information relating to a time of sending the skin image from the second user terminal; a step for storing the first skin image in the image storage after associating the source information and the time information with the first skin image; a step for taking out the stored first skin image to diagnose the first skin image for the skin lesion with the use of the first diagnosis program; a step for storing a diagnosis result for the first skin image in the diagnosis result storage after associating the source information and the time information with the diagnosis result; a step for receiving a second skin image of the pigmentary deposition portion same as the first skin image picked up by the camera device with dermoscope at a different time point, source information for identifying the pigmentary deposition portion, and time information relating to a time of sending the skin image from the second user terminal; a step for storing the second skin image after associating the source information and the time information with the second skin image; a step for taking out the stored second skin image to diagnose the second skin image for the skin lesion with the use of the first diagnosis program; a step for diagnosing the skin lesion based on a comparison between the diagnosis result for the first skin image and a diagnosis result for the second skin image with the use of the second diagnosis program; and a step for sending at least one of the diagnosis results of the first diagnosis program and the second diagnosis program to a destination designated by the source information.

A program for causing a computer to execute a remote diagnosis for diagnosing a skin lesion, the computer being communicably connected to a user terminal provided with a camera device with dermoscope and comprising a diagnosis program for diagnosing skin images for the skin lesion and an image storage for storing the skin images to be diagnosed, wherein the program may cause the computer to execute a step for receiving a skin image picked up by the camera device with dermoscope and source information for identifying the user terminal with the dermoscope; a step for storing the received skin image in the image storage after associating the source information with the skin image; a step for taking out the skin image stored in the image storage and diagnosing the skin image for the skin lesion with the use of the diagnosis program; and a step for sending a diagnosis result to a destination designated by the source information.

The program according to this invention is a program for causing a computer to execute a remote diagnosis for diagnosing a skin lesion, the computer being communicably connected to a user terminal provided with a camera device with dermoscope and comprising a first diagnosis program which diagnoses skin images for the skin lesion; a second diagnosis program for diagnosing the skin lesion based on plural diagnosis results for the skin lesion; an image storage for storing the skin images to be diagnosed; and a diagnosis result storage for storing a diagnosis result, wherein the program may cause the computer to execute a step for receiving a first skin image of a pigmentary deposition portion picked up by the camera device with dermoscope, source information for identifying the pigmentary deposition portion, and time information relating to a time of sending the skin image; a step for storing the first skin image in the image storage after associating the source information and the time information with the first skin image; a step for taking out the first skin image stored in the image storage to diagnose the first skin image for the skin lesion with the use of the first diagnosis program; a step for storing a diagnosis result for the first skin image in the diagnosis result storage after associating the source information and the time information with the diagnosis result; a step for receiving a second skin image of the pigmentary deposition portion same as the first skin image picked up by the camera device with dermoscope, source information for identifying the second pigmentary deposition portion, and time information relating to a time of sending; a step for storing the received second skin image in the image storage after associating the source information and the time information with the second skin image; a step for taking out the second skin image stored in the image storage to diagnose the second skin image for the skin lesion with the use of the first diagnosis program; a step for diagnosing the skin lesion based on a comparison between the diagnosis result for the first skin image and a diagnosis result for the second skin image with the use of the second diagnosis program; and a step for sending at least one of the diagnosis results of the first diagnosis program and the second diagnosis program to a destination designated by the source information.

A storage of this invention is a storage storing any one of the above-described programs in a machine-readable form.

The remote diagnosis apparatus according to this invention is a remote diagnosis apparatus communicably connected to a user terminal provided with a camera device with dermoscope, the remote diagnosis apparatus comprising: a first diagnosis program for diagnosing skin images for the pigmentary deposition and a second diagnosis program for diagnosing based on plural diagnosis results for the pigmentary deposition and receiving a first skin image of a pigmentary deposition portion picked up by the camera device with dermoscope from the user terminal; diagnosing the received first skin image for the pigmentary deposition with the use of the first diagnosis program; storing a diagnosis result for the first skin image; receiving a second skin image of the pigmentary deposition portion same as the first skin image picked up by the camera device with dermoscope at a different time point from the user terminal; diagnosing the second skin image for the pigmentary deposition with the use of the first diagnosis program; comparing the diagnosis result for the first skin image with a diagnosis result for the second skin image with the use of the second diagnosis program; and sending at least one of the diagnosis results of the first diagnosis program and the second diagnosis program to a destination designated by the source information.

The remote diagnosis apparatus according to this invention may be a remote diagnosis apparatus communicably connected to a user terminal provided with a camera device with dermoscope, the remote diagnosis apparatus comprising: a first diagnosis program diagnosing skin images for the pigmentary deposition; a second diagnosis program for diagnosing based on plural diagnosis results for the pigmentary deposition; an image storage for storing the skin images to be diagnosed; and a diagnosis result storage for storing a diagnosis result, receiving a first skin image of a pigmentary deposition portion picked up by the camera device with dermoscope, source information for identifying the pigmentary deposition portion, and first time information relating to a time of sending the first skin image from the user terminal, storing the first skin image in the image storage after associating the source information and the first time information with the first skin image, taking out the first skin image stored in the image storage to diagnose the first skin image for the pigmentary deposition with the use of the first diagnosis program, storing a diagnosis result for the first skin image in the diagnosis result storage after associating the source information and the first time information with the diagnosis result, receiving a second skin image of the pigmentary deposition portion same as the first skin image picked up by the camera device with dermoscope at a different time point, source information for identifying the pigmentary deposition portion, and second time information relating to a time of sending the skin image from the user terminal, storing the received second skin image in the image storage after associating the source information with the second skin image, taking out the stored second skin image to diagnose the second skin image for the pigmentary deposition with the use of the first diagnosis program, diagnosing through a comparison between the diagnosis result for the first skin image and a diagnosis result for the second skin image with the use of the second diagnosis program, and sending at least one of the diagnosis results of the first diagnosis program and the second diagnosis program to a destination designated by the source information.

The remote diagnosis apparatus according to this invention may be a remote diagnosis apparatus communicably connected to a user terminal provided with a camera device with dermoscope, the remote diagnosis apparatus comprising: a receiving unit for receiving a skin image of a pigmentary deposition portion picked up by the camera device with dermoscope, source information for identifying the pigmentary deposition portion, and time information relating to a time of sending the skin image from the user terminal; an image storage for storing the received skin image after associating the source information and the time information with the skin image; a first diagnosis program for taking out the skin image stored in the image storage to diagnose the skin image for a pigmentary deposition; a diagnosis result storage for storing a diagnosis result after associating the source information and the time information with the diagnosis result for the skin image; a second diagnosis program capable of diagnosing the pigmentary deposition based on plural diagnosis results for the pigmentary deposition of the skin image, which are stored in the diagnosis result storage and different in time information; and a sending unit for sending at least one of diagnosis results of the first diagnosis program and the second diagnosis program to a destination designated by the source information.

Note that the user terminal in each of the remote diagnosis apparatuses may preferably be provided with a portable telephone function. Also, the user terminal may preferably communicate via the Internet.

The remote diagnosis system according to this invention is a remote diagnosis system for diagnosing a skin lesion, comprising a user terminal provided with a camera device with dermoscope and a remote diagnosis apparatus communicably connected to the user terminal and comprising a first diagnosis program for diagnosing skin images for the pigmentary deposition and a second diagnosis program capable of diagnosing the pigmentary deposition based on a comparison of plural results of the diagnosis for the pigmentary deposition, wherein the user terminal sends a first skin image of a pigmentary deposition portion picked up by the camera device with dermoscope to the remote diagnosis apparatus; the remote diagnosis apparatus receives the first skin image and diagnoses the received first skin image for the pigmentary deposition with the use of the first diagnosis program; the user terminal sends a second skin image of the pigmentary deposition portion same as the first skin image picked up by the camera device with dermoscope to the remote diagnosis apparatus; the remote diagnosis apparatus receives the second skin image; diagnoses the received second skin image for the pigmentary deposition with the use of the first diagnosis program; diagnosing the pigmentary deposition based on a comparison between the diagnosis result for the first skin image with a diagnosis result for the second skin image with the use of the second diagnosis program; and sends at least one of the diagnosis results of the first diagnosis program and the second diagnosis program to the second user terminal.

The remote diagnosis system according to this invention may be a remote diagnosis system for diagnosing a pigmentary deposition, comprising: a user terminal provided with a camera device with dermoscope and a remote diagnosis apparatus communicably connected to the user terminal, wherein the user terminal comprises a sending unit for sending a skin image of a pigmentary deposition portion picked up by the camera device with dermoscope, source information for identifying the pigmentary deposition portion, and time information relating to a time of sending the skin image to the remote diagnosis apparatus, and the remote diagnosis apparatus comprises: an image receiving unit for receiving the skin image, the source information, and the time information from the second user terminal; an image storage for storing the skin image after associating the source information and the time information with the skin image; a first diagnosis program for diagnosing the received skin image for the pigmentary deposition; a second diagnosis program capable of diagnosing based on plural diagnosis results for the pigmentary deposition; a diagnosis result storage for storing the diagnosis result; and a sending unit for sending the diagnosis result to a predetermined destination.

Note that the user terminal in each of the remote diagnosis systems may preferably be provided with a portable telephone function. Also, the user terminal may preferably communicate via the Internet.

A remote diagnosis method according to this invention may be a remote diagnosis method for diagnosing a pigmentary deposition other than skin lesions using a remote diagnosis apparatus which is communicably connected to a user terminal provided with a camera device with dermoscope and comprises: an image receiving unit for receiving a skin image to be diagnosed from the user terminal; an image storage for storing a skin image; a first diagnosis program for diagnosing skin images for the pigmentary deposition; a second diagnosis program diagnosing based on plural diagnosis results for the pigmentary deposition; a diagnosis result storage for storing the diagnosis results; and a diagnosis result sending unit for sending at least one of the diagnosis results of the first diagnosis program and the second diagnosis program to the user terminal, wherein the image receiving unit receives a first skin image of a pigmentary deposition portion picked up by the camera device with dermoscope, source information for identifying the pigmentary deposition portion, and first time information relating to a time of sending the first skin image from the user terminal; the image storage stores the first skin image after associating the source information and the first time information with the skin image; the first diagnosis program takes out the first skin image stored in the image storage to diagnose the first skin image for the pigmentary deposition; the diagnosis result storage stores a diagnosis result for the first skin image after associating the source information and the first time information with the diagnosis result; the image receiving unit receives a second skin image of the pigmentary deposition portion same as the first skin image picked up by the camera device with dermoscope at a different time point, source information for identifying the pigmentary deposition portion, and second time information relating to a sending time from the user terminal; the diagnosis result storage stores the received second skin image after associating the source information with the second skin image; the first diagnosis program takes out the stored second skin image to diagnose the second skin image for the pigmentary deposition; the second diagnosis program diagnoses through a comparison between the diagnosis result for the first skin image and the diagnosis result for the second skin image; the diagnosis result sending unit sends at least one of the diagnosis results of the first diagnosis program and the second diagnosis program to a destination designated by the source information.

The diagnosis program according to this invention is a diagnosis program which is updated based on plural skin images stored in a database of which diagnoses have been determined and used for diagnosing skin images to be diagnosed for a skin lesion, the diagnosis program causing a computer to execute a step for performing image processing by separating a pigmentary disorder portion of a skin lesion from a peripheral normal portion in each of plural skin images on which the diagnoses have been made and separating a rim portion of the separated pigmentary disorder portion; a step for deciding parameters to be used for discrimination of characteristics of the pigmentary disorder, such as a color, a texture, an asymmetricity, and a circularity; and a step for performing evaluation of each of the parameters using a predetermined system on the diagnosed skin images with the use of each of the parameters, performing evaluation of each of other parameters using the predetermined system on the diagnosed skin images with the use of each of other parameters, repeating the same operation until a diagnosis capability of any one of parameter combinations reaches a predetermined value, and, ultimately, selecting a parameter combination having a smallest number of parameters from the parameter combinations having the diagnosis capability equal to or more than the predetermined value. As used herein, the diagnosis capability means a value obtained by a function obtained by combining sensitivity, specificity, an average value thereof, and the like, and the predetermined value when the value of the case of perfectly correct diagnosis is 100% may be, but not limited to, 70% or more, preferably 80% or more, and more preferably 90% or more.

The diagnosis program may further cause the computer to execute a step for performing the image processing on a skin image to be diagnosed and a step for obtaining a diagnosis result for the skin image to be diagnosed using the predetermined system with the use of the parameter combination selected in the fourth step.

In the above-described diagnosis programs, the predetermined system may be, but not limited to, a neural network system. In the above-described diagnosis programs, at least one of the parameter evaluations may be performed by employing a leave-one-out method. The leave-one-out method is preferred when the number of skin images on which diagnoses are determined is small, but the evaluation method is not limited to the method. In the above-described diagnosis programs, the skin lesion which is a subject of the diagnosis may be melanoma.

A storage according to this invention is a storage storing the above-described program in a machine-readable form.

Further, a method for screening a cosmetic agent or a drug for diminishing a pigmentary deposition of skin may use the above-described remote diagnosis apparatus and the remote diagnosis system having the diagnosis program for diagnosing the pigmentary deposition or may use the remote diagnosis method for pigmentary deposition other than skin lesion.

### CROSS REFERENCE TO RELATED PUBLICATIONS

This invention claims priority based on Japanese Patent Application No. 2004-4747 filed on Jan. 9, 2004, which is incorporated herein by reference.

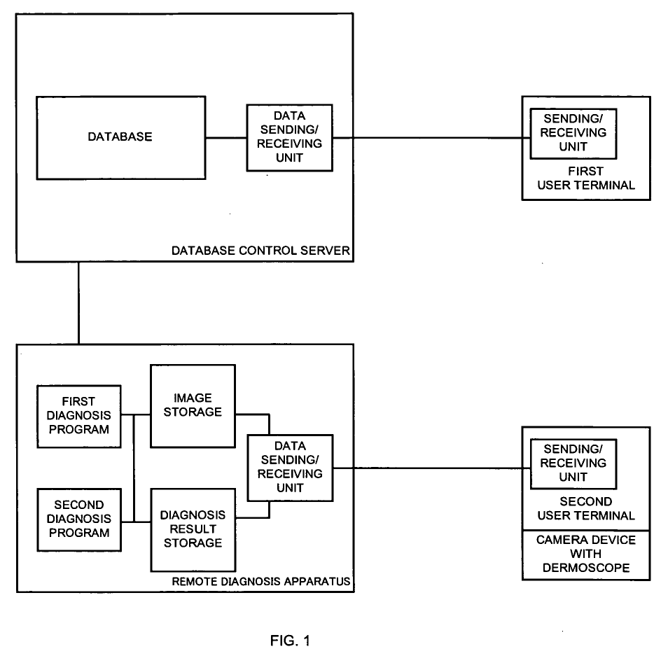

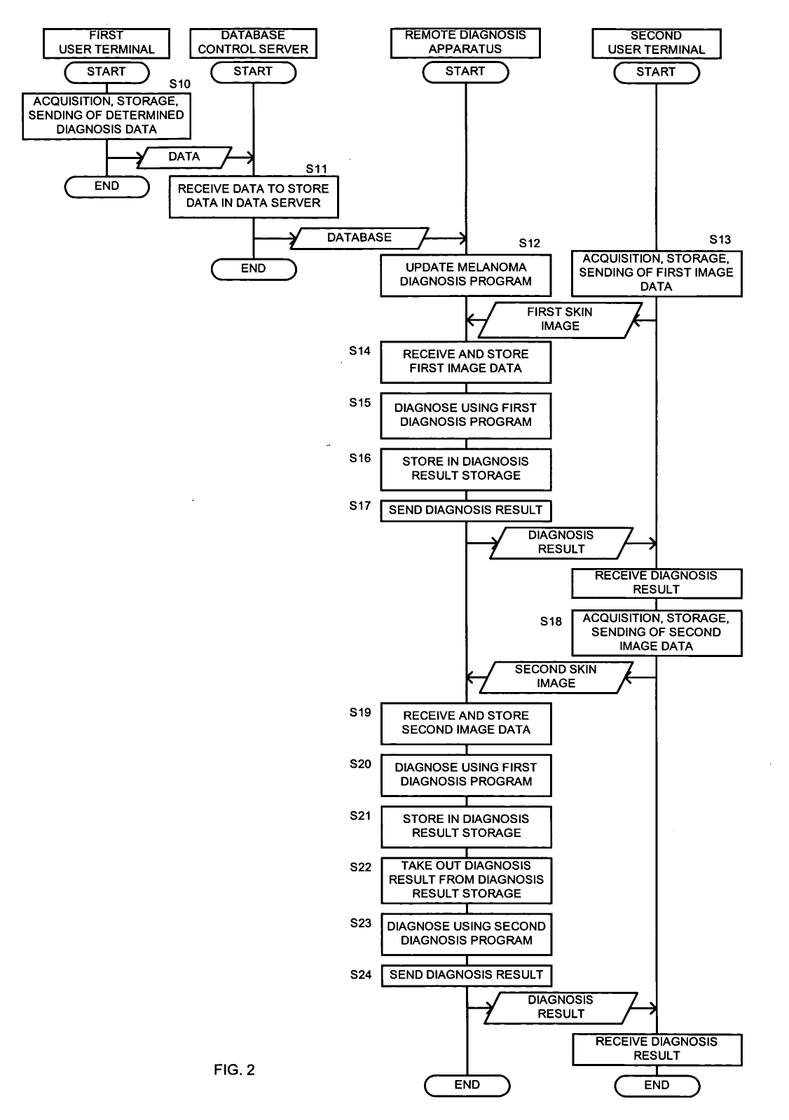

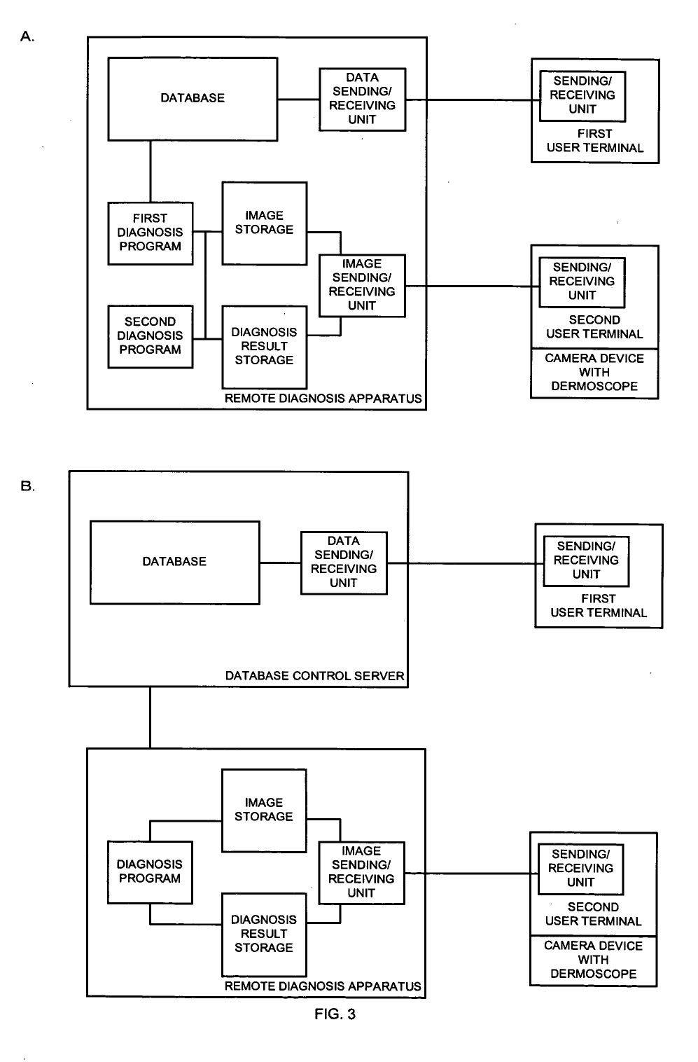

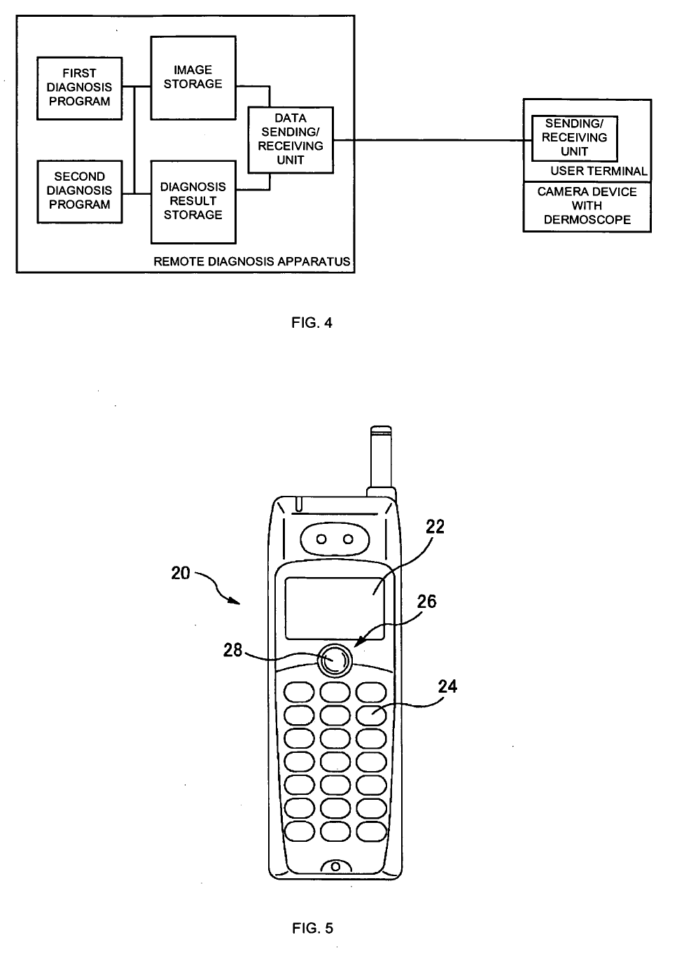

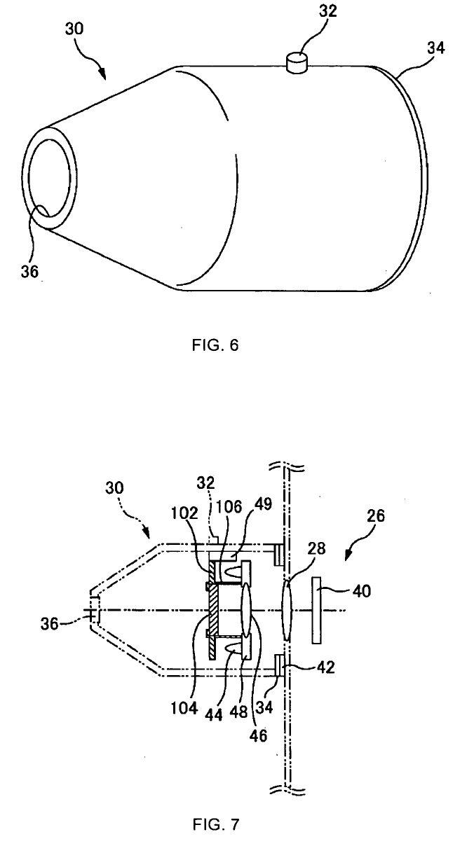

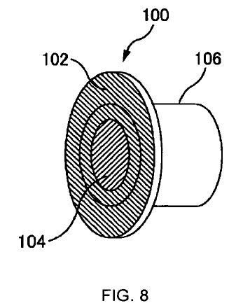

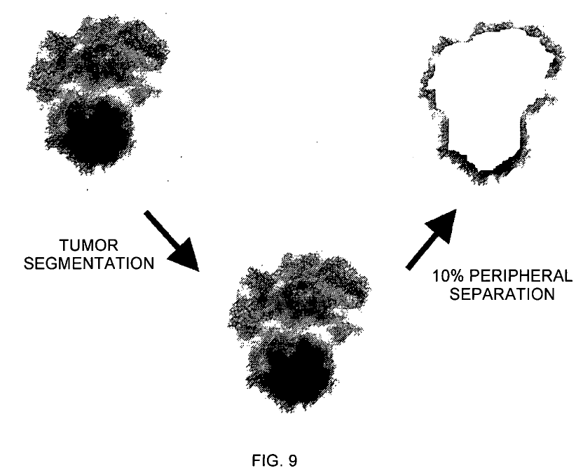

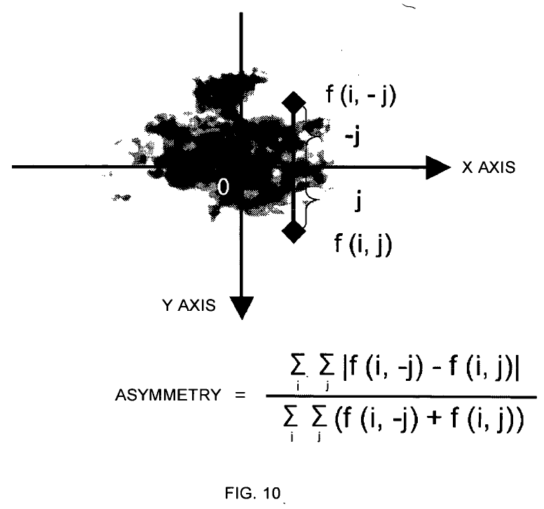

### BRIEF DESCRIPTION OF THE DRAWINGS

[FIG. 1] A block diagram of a melanoma remote diagnosis system according to Embodiment I of the remote diagnosis system of this invention.

[FIG. 2] A flowchart showing one example of operation of each of component parts of the melanoma remote diagnosis system according to Embodiment I of the remote diagnosis system of this invention.

[FIG. 3] A block diagram showing another embodiment of the remote diagnosis system according to this invention.

[FIG. 4] A block diagram showing a pigmented macule remote diagnosis system according to Embodiment II of the remote diagnosis system of this invention.

[FIG. 5] A diagram showing an appearance of one example of portable telephone with camera according to the embodiments of this invention.

[FIG. 6] A diagram showing an appearance of one example of image pickup device of the portable telephone with camera according to the embodiments of this invention.

[FIG. 7] A sectional view showing one example of interior of a unit of the portable telephone with camera according to the embodiments of this invention.

[FIG. 8] A diagram showing one example of polarizing filter of the portable telephone with camera according to the embodiments of this invention.

[FIG. 9] A diagram showing one example of image processing with a diagnosis program according to the embodiments of this invention.

[FIG. 10] A diagram and a calculation formula showing one example of asymmetricity determination method of the diagnosis program according to the embodiments of this invention.

### BEST MODE FOR CARRYING OUT THE INVENTION

Hereinafter, embodiments of this invention will be described in detail in conjunction with examples. In the case of using a commercially available measurement device, a protocol attached to the measurement device is used unless otherwise explained.

Object, characteristics, advantages, and ideas of this invention are clearly understood by those skilled in the art by description of this specification, and those skilled in the art can easily realize this invention from the description of this specification. Embodiments and specific examples of this invention described below are given for the purposes of indicating preferable modes of this invention, exemplification, or explanation and not for the purpose of limiting this invention to them. It is apparent for those skilled in the art that various alterations and modifications can be made based on the description of this specification within the spirit and scope of this invention disclosed in this specification.

Examples of the skin lesion to be the subject of the diagnosis system according to this invention include malignant tumors such as melanoma and basal cell cancer, and it is possible to use the diagnosis system for distinguishing the malignant skin diseases from benign skin diseases such as benign nevus pigmentosus and senile wart as well as for follow-up after removal of the malignant skin diseases. Also, it is possible to use the remote diagnosis system according to this invention for diagnosis of pigmented macule (so-called blot), such as confirmation of effect of pigmented macule removal surgery before and after the surgery and confirming effects of cosmetic agents and drugs for diminishing the pigmented macule and pigmentary deposition on a specific portion (e.g. mammilla) of the body. It is possible to use the effects for a screening of the cosmetic agents and drugs for diminishing the pigmentary deposition.

### EMBODIMENT 1

### Melanoma Remote Diagnosis System

As shown in the block diagram of FIG. 1, the remote diagnosis system of this embodiment is a remote diagnosis system for diagnosing presence/absence and a disease stage of melanoma, comprising a first user terminal, a database control server communicably connected to the first user terminal, a second user terminal provided with a camera device with dermoscope, and a remote diagnosis apparatus communicably connected to the database control server and the second user terminal. The first user terminal is provided with a data sending/receiving unit for sending/receiving data such as an image to/from the database control server, and the database control server is provided with a data sending/receiving unit for sending/receiving data such as an image to/from the first user terminal and a database for storing data which are received from the first user terminal and relate to a skin lesion. The second user terminal is provided with a data sending/receiving unit for sending/receiving data such as an image and a diagnosis result to/from the remote diagnosis apparatus, and the remote diagnosis apparatus is provided with a data sending/receiving unit for sending/receiving data such as a skin image and a diagnosis result to/from the second user terminal, an image storage serving as image storage means, a diagnosis result storage serving as diagnosis result storage means, a first diagnosis program for diagnosing a skin image for a skin lesion, a second diagnosis program for diagnosing the skin lesion based on plural diagnosis results for the skin lesion, and a data sending unit for sending data such as a diagnosis result to a predetermined receiver.

With the use of the remote diagnosis system shown in FIG. 1, remote diagnosis of melanoma is performed by process steps described below, for example. Flowcharts corresponding to the diagnosis process steps are shown in FIG. 2.

(1) To start with, a medical professional such as a doctor pathologically diagnoses a patient having a skin lesion to send data such as a picture of the skin lesion and a determined diagnosis result with the use of the first user terminal such as a personal computer to the database control server (S10). As described above, since the first user terminal has the role of sending the image and the data of the skin lesion after determination of the diagnosis to the database control server, the first user terminal may preferably be a terminal such as a personal computer possessed by a medical professional such as a doctor. The data sent from the first user terminal include a skin image of skin lesion, characteristics of skin lesion (site in the body, color, size, state, etc.), determined diagnosis of skin lesion (if the skin lesion is melanoma or not; type of melanoma in the case where the skin lesion is melanoma, etc.), and the like.

(2) The database control server receives the data relating to the skin lesion from the first user terminal and then stores the data in the database for storing the data (S11).

The database is used as basic data when deciding parameters in the first diagnosis program as described later in this specification, and it is possible to increase accuracy of the diagnosis of the diagnosis program described later in this specification by using a large size database. Therefore, the data relating to the skin lesion may be collected by doctors belonging to a hospital where the database control server is placed, but the data may preferably be collected via the Internet from doctors belonging to hospitals nationwidely or internationally.

Also, it is possible to update the first diagnosis program when so required based on the data stored in the database (S12). For instance, the remote diagnosis apparatus may request the database control server for the database once a week to obtain the latest database in which data are updated, thereby updating the first diagnosis program based on the data.

(3) The patient takes a picture of a skin lesion such as nevus pigmentosus which might be melanoma through the dermoscope using the second user terminal such as a portable telephone with camera to which the dermoscope with a polarizing filter is attached by an adapter and then stores the skin image in the portable telephone (S13). The patient accesses the Internet using an Internet access function of the portable telephone to send the picked up skin image to the remote diagnosis apparatus with the use of an e-mail program or a browser (S13).

When sending the skin image to the remote diagnosis apparatus, an appropriate ID is assigned to the skin lesion as source information for identifying the skin lesion to be sent together with the image. Also, time information relating to the time of sending is attached to the image. Also, in the case where the patient desires that a diagnosis result is sent to a user terminal different from the portable telephone or the like used for sending the image, an e-mail address of a desired destination of the diagnosis result may be attached to the image so that the user terminal is registered. Such registration of desired destination enables also to deal with cases when the desired destination is changed after sending the image.

Though the second user terminal may be a personal computer, a web-enabled game console, or a portable telephone insofar as it is possible to communicate with the remote diagnosis apparatus, the portable telephone capable of communicating via the Internet is preferred in view of the populization rate and portability. More specifically, since the portable telephone with camera is widely used at present, anybody can access and use the diagnosis system conveniently when the person desires to know if a newly found nevus pigmentosus is melanoma. However, since it is impossible to take a proper picture for the use in diagnosis of cells such as the melanoma existing in the skin by an ordinary camera incorporated into portable telephones, the dermoscope will be required.

The dermoscope is a microscope developed for observing skin lesions, and it is possible to conduct the observation through the skin by eliminating light reflected from the skin surface. One type of the dermoscope is used in direct contact with the skin after applying on the skin a gel used for sonographic diagnosis, and the other type has a polarizing filter incorporated in the dermoscope. As the dermoscope to be used in this invention, the latter type may be preferred since anybody including ordinary people without doctor's license can use such dermoscope easily. By the incorporation of the polarizing filter, it is possible to conveniently use the dermoscope particularly the dermoscope attached to the portable telephone.

In order to provide the user terminal such as the portable telephone with the dermoscope, a built-in type or external connection type using SCSI, USB, firewire, or the like may be used. In the case of the portable telephone with camera, the dermoscope may preferably be formed into an attachable/detachable type by taking advantage of the camera so that the dermoscope is attachable to portable telephones of various manufacturers by means of an adapter. It is considered that magnifying power and color reproducibility are different depending on the terminals, a color sample pallet and a scale bar to be used for color correction and identification of the magnifying power at a later date may be installed into the dermoscope to be displayed in the skin image.

(4) The remote diagnosis apparatus receives the skin image of the skin lesion picked up by the camera device with dermoscope, the skin lesion ID, and the time information from the second user terminal to store the skin image in the image storage after associating the skin lesion ID and the time information with the skin image (S14). A melanoma diagnosis program serving as the first diagnosis program takes out skin images in the order of receipt from the storage to determine if the skin lesion is melanoma and a disease stage when the skin lesion is melanoma (S15). The thus-obtained diagnosis result is stored in the diagnosis result storage (S16).

Examples of the image storage and the diagnosis result storage include but not limited to, a memory, a cache, a hard disk, a removable memory and the like.

As described above, by providing the remote diagnosis apparatus with the storage storing the diagnosis program for judging from the skin image data if the skin lesion is melanoma, etc. or normal and a degree of severity when the skin lesion is melanoma, it is possible to analyze the skin image automatically to diagnose skin lesions.

In the case where a doctor diagnoses with the use of a dermoscope, the diagnosis may be subjective, and the diagnosis result may differ depending on doctors in many cases. In this invention, since the dermoscope is used in combination with the diagnosis program executed by the computer, it is possible to use the dermoscope for more objective diagnoses. Also, it is possible to perform the process steps from the step of picking up image to the step of making diagnosis automatically.

(5) The remote diagnosis apparatus sends the diagnosis result stored in the diagnosis result storage to the predetermined destination designated by the skin lesion with the use of an e-mail program or a browser (S17). The destination may be the second user terminal which sent the skin image or the desired destination which was registered when the skin image was sent and is different from the second user terminal.

(6) Further, another diagnosis for the same skin lesion may be requested after the first diagnosis. The patient takes a picture of the skin lesion in the same manner as in the first time and stores the thus-obtained second skin image in the portable telephone to send the skin image to the remote diagnosis apparatus (S18). In this case, it is unnecessary to use the user terminal which is the same as that of the first time, but the ID for identifying the skin lesion must be the same as that of the first time. With the use of the same ID, the remote diagnosis apparatus recognizes the skin image as that of the identical skin lesion which is identified by the sent ID. Also, an e-mail address of a desired destination of a diagnosis result is attached to the skin image. It is unnecessary to use the desired destination same as that of the first time. Also, second time information indicating the time of sending, which is different from that of the first time, is attached when sending the second skin image.

(7) The remote diagnosis apparatus receives the second skin image of the skin lesion picked up by the camera device with dermoscope, the skin lesion ID, and the second time information to store the second skin image in the image storage after associating the skin lesion ID and the second time information with the skin image (S19). The melanoma diagnosis program serving as the first diagnosis program takes out skin images in the order of receipt from the image storage to determine if the skin lesion is melanoma and a disease stage when the skin lesion is melanoma (S20). The thus-obtained diagnosis result is stored in the diagnosis result storage (S21).

(8) A comparison diagnosis program provided in the remote diagnosis apparatus and serving as the second diagnosis program obtains the diagnosis result for the first skin image and the diagnosis result for the second skin image from the diagnosis result storage to make a further diagnosis on the skin lesion based on a comparison with the diagnosis result for the second skin images (S23). Thus, it is possible to know a change of the skin lesion from the time point of sending the first skin image to the time point of sending the second skin image. For instance, in the case of the melanoma in this embodiment, it is possible to measure the size of the melanoma using the melanoma diagnosis program serving as the first diagnosis program, and it is judged that the melanoma is malignant when there is an increase in size as time advances.

(9) The remote diagnosis apparatus sends the diagnosis result obtained by the first diagnosis program on the second skin image and the diagnosis result obtained by the second diagnosis program to the predetermined destination designated by the skin lesion with the use of a mail software or a browser. The diagnosis results are typically returned to the second user terminal which was used for sending the skin image to be diagnosed (S24).

Foregoing is detailed description of one example of embodiment according to this invention. Though the database having data relating to skin lesion is provided in the database control server in the foregoing embodiment, the database may be provided in the remote diagnosis apparatus and a second user terminal of the doctor may be communicable with the remote diagnosis apparatus, so that the doctor directly sends data relating to the skin lesion to the remote diagnosis apparatus (see, for example, FIG. 3A). Also, in the case of not tracing time course, the comparison diagnosis program serving as the second diagnosis program may be eliminated (see, for example, FIG. 3B). Also, the database control and diagnosis program update may be performed manually as required. In the case of not performing the diagnosis program update, the database control server and constitutions relating to the database control server may not be necessary. A constitution wherein the storage is not required is also considered. Also, a combination of the above constitutions may be utilized.

### EMBODIMENT II

### Pigmented Macule Diagnosis System

As shown in the block diagram of FIG. 4, the remote diagnosis system in this embodiment is a remote diagnosis system for diagnosing pigmented macule and provided with a user terminal provided with a camera device with dermoscope and a remote diagnosis apparatus to be communicated with the user terminal. The user terminal is provided with a sending/receiving unit for sending/receiving data such as an image, a diagnosis result, and the like to/from the remote diagnosis apparatus, and the remote diagnosis apparatus is provided with a sending/receiving unit for sensing/receiving data such as a skin image, a diagnosis result, and the like to/from the user terminal, an image storage, a diagnosis result storage, a first diagnosis program for diagnosing a skin image for a skin lesion, a second diagnosis program for diagnosing the skin lesion based on plural diagnosis results for the skin lesion, and a data sending unit for sending data such as a diagnosis result to a predetermined receiver.

With the use of the remote diagnosis system as shown in FIG. 4, a remote diagnosis of pigmented macule is performed by process steps described below, for example. In this embodiment, remote diagnosis is utilized to examine the effects of using cosmetic agents and drugs for diminishing the pigmented macule (blot) of skin for cosmetic purposes.

A person who desires a diagnosis on a pigmented macule obtains a first skin image of the pigmented macule using the user terminal such as a portable telephone with camera which is provided with a dermoscope with polarizing filter and sends the first skin image to the remote diagnosis apparatus in the same manner as in Embodiment I-(3). The remote diagnosis apparatus diagnoses the pigmented macule with the use of the diagnosis program and stores the diagnosis result in the diagnosis result storage in the same manner as in Embodiment I-(4). In the case of the pigmented macule, a diagnosis result relating particularly to color strength among elements to be judged by the first diagnosis program is used for the diagnosis. Next, after using a cosmetic agent for a test, the user terminal picks up a second image of the identical pigmented macule to send the second image to the remote diagnosis apparatus in the same manner as in Embodiment I-(6). The remote diagnosis apparatus uses the first diagnosis program to obtain a diagnosis result for the second skin image in the same manner as in Embodiment I-(7) and judges if the color of the pigmented macule has been diminished based on a comparison between the diagnosis result for the first skin image and the diagnosis result for the second skin image with the use of the comparison diagnosis program in the same manner as in Embodiment I-(8). The judgment result is sent to the predetermined destination designated by the pigmented macule in the same manner as in Embodiment I-(9). The diagnosis result is typically returned to the user terminal which sent the skin image to be diagnosed.

In addition, this embodiment is used for a diagnosis of a color of a skin of a specific part (e.g. mammilla) of the body without limitation to the skin lesion such as pigmented macule.

Dermoscope with Polarizing Filter for Portable Telephone

As described in the foregoing, the dermoscope used in this embodiment may preferably be provided with the polarizing filter and most preferably be attached to the camera incorporated into the portable telephone by means of an adapter. Hereinafter, one example of the dermoscope will be described in detail.

An appearance of one example of the portable telephone 20 with camera is shown in FIG. 5. In the portable telephone 20, a camera 26 is disposed under a display 22 and above push-buttons 24. The camera 26 is provided with an objective lens 28 and an image pickup device 40 shown in FIG. 6. The image pickup device 40 performs imaging by image light introduced from an object to be picked up via the objective lens 28 and is formed of a CCD in this embodiment. In the case of picking up an image by using this portable telephone 20 alone, it is possible to pick up a reduced image.

A unit 30 serving as the adapter to be attached to the portable telephone 20 is shown in FIG. 6. The unit 30 is attached to the portable telephone 20 to form an image picked up by the image pickup device 40 into an enlarged image. A horizontal sectional view of a state in which the unit 30 is attached to the portable telephone 20 is shown in FIG. 4. In a state where the unit 30 is coaxial with an optical axis of the objective lens 28 included in the camera 26 of the portable telephone 20, a first member 42 is attached to a surface of the portable telephone 20 to fix a second member 34 in such a manner as to enclose the exposed objective lens 28, thereby realizing attachment in a state where the unit 30 is detachable from the portable telephone 20. For instance, the unit 30 may be fixed by fitting the unit 30 into a projection formed on one of the members and a depression formed on the other member. Also, the unit 30 may be fixed by a magnetic force by forming one of the members from a metal plate and forming the other member from a magnet.

As shown in FIG. 7, a mount 48 is fixed on an inner periphery of the unit 30 inside the unit 30. The mount 48 is a circular plate having an outer diameter identical to an inner diameter of the unit 30 and a circular hole formed at the center. A magnifying lens 46 is fitted into the hole of the mount 48 to be attached to the unit 30 via the mount 48.

In a state where the unit 30 is fixed to the portable telephone 20, a distance from a unit tip part 36 to an outer surface of the portable telephone 20 is such that a point of focus by a combination of the objective lens 28 and the magnifying lens 46 is kept within a range corresponding to an imaging range of the object to be picked up when the tip part 36 is brought into contact with the object to be picked up. Therefore, by bringing the tip part 36 into contact with the object to be picked up, the camera is automatically focused on a part of the object to be picked up enclosed by the tip part 36.

Six light sources 44, for example, are disposed on a front face of the mount 48. Each of the light sources may be a small LED and illuminates the imaging range of the object to be picked by emitting light.

A battery 52 serving as a power source is provided on the inner periphery of the unit 30 and near the mount 48. The battery 49 provides energy for the light emission of the light sources 44. A switch 32 is disposed on the surface of the unit 30 to control a state of light emission of the light sources 44 by changing a connection state between the battery 49 and the light sources 44.

A polarizing filter device 100 shown in FIG. 8 is disposed in front of the mount 48. The device 100 is provided with a light source polarizing filter 102 for polarizing light from the light sources 44, a received light polarizing filter 104 for polarizing light from the object to be picked up, a light shielding tube 106 for shielding light so that light exiting from the light sources through the light source polarizing filter 102 to outside the unit 30 is not blended with light entering the magnifying lens 46 through the received light polarizing filter 104 from the outside. Each of the received light polarizing filter 104 and the light source polarizing filter 102 has a polarizing filter, and the polarizing filters differ in polarizing direction.

With the use of the portable telephone with camera 20 and the unit 30 mounted on the portable telephone 20 having the above-described constitutions, a skin which is an object to be picked up is picked up using the camera function incorporated into the portable telephone 20 in a state where the tip part 36 of the unit 30 being contacted with the skin. The light emitted from the light sources 44 passes through the light source polarizing filter 102 to be light polarized in the predetermined direction. Since the light reflected by the skin surface has an oscillation direction same as that of the light from the light sources 44, the light is eliminated as being prevented from passing through the received light polarizing filter 104 having the different polarizing direction. In turn, since the light reflected inside the skin has various polarizing directions, the light having an oscillation direction same as that of the received light polarizing filter 104 passes through the received light filter 104.

With the use of the polarizing filter device 100, it is possible to effectively eliminate the light reflected by the skin surface and to take a beautiful picture of melanoma existing under the skin.

Further, the use of the portable telephone to which the unit with polarizing filter is attached for the remote diagnosis enables ordinary people without medical knowledge to pick up a beautiful skin image of a skin lesion to facilitate diagnoses on melanoma and the like.

Melanoma Diagnosis Program

This diagnosis system is applicable to various skin lesions that can be diagnosed through examination of the skin surface and capable of using a diagnosis program appropriate for each of the skin lesions.

In the case of creating a diagnosis program, plural skin images on which diagnoses are determined are used as basic data to decide parameters to be obtained by a skin image in such a manner as to achieve the maximum diagnosis accuracy. A diagnosis of a skin image which is a diagnosis subject on which a diagnosis has not been determined is performed by using the decided parameters. Therefore, since the program is improved as the number of determined diagnoses and the number of inputted basic data is increased, it is recommended to decide a new diagnosis parameter by updating the diagnosis program sequentially with the use of the database to which new basic data has been inputted.

However, since a premise that a skin lesion has a pattern is included in the decision of the diagnosis program, the diagnosis accuracy is deteriorated if a skin lesion having an exceptional pattern is used as the basic data. Therefore, in the case where the parameters are changed by a large degree or the diagnosis accuracy is deteriorated by a large degree when the diagnosis program is updated after adding new data as the basic data, such added data may be eliminated as an error.

Since it is possible to eliminate indeterminate elements for diagnosis result, which can be caused depending on doctor's experience of skin lesion diagnosis, by using the diagnosis program, the diagnosis result with reliable probability is obtained.

Hereinafter, a melanoma diagnosis program will be descried as one example.

<Image Processing>

To start with, a pigmentary disorder portion is separated from a peripheral normal portion. This processing is performed automatically by using an algorithm having an automatic threshold value selection method based on a least square method employed after Gaussian filtering and Laplacian filtering. Next, a rim portion having a width which is 10% of a diameter is specified on a circumference of the pigmentary disorder portion to be separated by employing the technique of erosion (FIG. 9).

<Calculation Processing>

Values (parameters) of a color, a texture, asymmetricity, circularity, and the like were used for discriminating the pigmentary lesion. Used as the values of the color were a minimum value, a maximum value, and an average value of each of red, green, and blue of each of the whole pigmentary disorder portion and the rim portion. The minimum value includes (1) a minimum value, (2) a minimum value corrected by the average value of the surrounding skin, and (3) a minimum value of a region of 5% or more of the whole region. A proportion of an area having an luminance of 100 or less was measured in both of the pigmentary disorder portion and the rim portion. Used as the values of the texture were standard deviation, skewness, uniformity, and energy of each of red, green, and blue in each of the pigmentary disorder portion and the rim portion. The asymmetricity is calculated from the expression shown in FIG. 10. The asymmetricity of each of a main axis and a secondary axis is calculated for each of red, green, blue, and binarized values.

The value of circularity c is defined by

c=P²/4πA

(in the equation, P represents a peripheral length of the rim portion, and A represents an area of the pigmentary disorder portion). With an increase in circularity, the pigmentary disorder portion becomes more elliptic to be complicated in shape.

As shown in Table 1, the number of parameters is 62, and the diagnosis program selects plural ones which are optimum for a diagnosis from the parameters. Hereinafter, one example of method of selecting the parameters in the diagnosis program will be described.

<Decision I of Parameters to be Used: Multivariate Stepwise Selection Discriminant Analysis>

Skin images of pigmentary disorder on skin surface obtained by the dermoscope with the polarizing filter are collected in view of the following conditions:

1) Absence of hair that shields a pigmentary disorder;
2) Elimination of a pigmentary disorder caused in extremity and mucous membrane; and
3) Inclusion of whole image of a pigmentary disorder.

Used in this embodiment were 23 cases of melanoma in situ (MIS), 36 cases of thin invasive melanoma (TIM) having a thickness of 0.75 mm or less, and 188 cases of Clark's nevi (CN).

With the use of the 62 parameters (see Table 1) in total, a multivariate stepwise discriminant analysis was performed to discriminate a difference between MIS and CN or between TIM and CN. SPSS (Windows, version 11.0.1 J; product of SPSS) was used for this analysis.

TABLE 1
Total parameters (62)

| Step | Parameter |
|---|---|
| 1 | Minimum value of red in overall tumor |
| 2 | Minimum value of green in overall tumor |
| 3 | Minimum value of blue in overall tumor |
| 4 | Maximum value of red in overall tumor |
| 5 | Maximum value of green in overall tumor |
| 6 | Maximum value of blue in overall tumor |
| 7 | Average value of red in overall tumor |
| 8 | Average value of green in overall tumor |
| 9 | Average value of blue in overall tumor |
| 10 | Deviation of red in overall tumor |
| 11 | Deviation of green in overall tumor |
| 12 | Deviation of blue in overall tumor |
| 13 | Skewness of red in overall tumor |
| 14 | Skewness of blue in overall tumor |
| 15 | Skewness of green in overall tumor |
| 16 | Energy of red in overall tumor |
| 17 | Energy of blue in overall tumor |
| 18 | Energy of green in overall tumor |
| 19 | Entropy of red in overall tumor |
| 20 | Entropy of blue in overall tumor |
| 21 | Entropy of green in overall tumor |
| 22 | Circularity (index of similarity to circle) |
| 23 | Ratio of black region having luminance of 100 |
| | or less in overall tumor |
| 24 | Ratio of black region having luminance of 100 |
| | or less in rim portion of tumor |
| 25 | Minimum value of red corrected by average |
| | value in normal skin |
| 26 | Minimum value of blue corrected by average |
| | value in normal skin |
| 27 | Minimum value of green corrected by average |
| | value in normal skin |
| 28 | Minimum value of red occupying 0.05% or more |
| | of area of overall tumor |
| 29 | Minimum value of blue occupying 0.05% or more |
| | of area of overall tumor |
| 30 | Minimum value of green occupying 0.05% or more |
| | of area of overall tumor |
| 31 | Longitudinal asymmetricity of red component |
| 32 | Longitudinal asymmetricity of blue component |
| 33 | Longitudinal asymmetricity of green component |
| 34 | Horizontal asymmetricity of red component |
| 35 | Horizontal asymmetricity of blue component |
| 36 | Horizontal asymmetricity of green component |
| 37 | Horizontal asymmetricity of shape irrelevant |
| | to color |
| 38 | Longitudinal asymmetricity of shape irrelevant |
| | to color |
| 39 | Minimum value of red in rim portion of tumor |
| 40 | Minimum value of green in rim portion of tumor |
| 41 | Minimum value of blue in rim portion of tumor |
| 42 | Maximum value of red in rim portion of tumor |
| 43 | Maximum value of green in rim portion of tumor |
| 44 | Maximum value of blue in rim portion of tumor |
| 45 | Average value of red in rim portion of tumor |
| 46 | Average value of green in rim portion of tumor |
| 47 | Average value of blue in rim portion of tumor |
| 48 | Deviation of red in rim portion of tumor |
| 49 | Deviation of green in rim portion of tumor |
| 50 | Deviation of blue in rim portion of tumor |
| 51 | Skewness of red in rim portion of tumor |
| 52 | Skewness of blue in rim portion of tumor |
| 53 | Skewness of green in rim portion of tumor |
| 54 | Energy of red in rim portion of tumor |
| 55 | Energy of blue in rim portion of tumor |
| 56 | Energy of green in rim portion of tumor |
| 57 | Entropy of red in rim portion of tumor |
| 58 | Entropy of blue in rim portion of tumor |
| 59 | Entropy of green in rim portion of tumor |
| 60 | Minimum value of red occupying 0.05% or more |
| | of area of rim portion of tumor |
| 61 | Minimum value of blue occupying 0.05% or more |
| | of area of rim portion of tumor |
| 62 | Minimum value of green occupying 0.05% or more |
| | of area of rim portion of tumor |

To start with, in order to minimize a λ value of Wilks, a forward selection or a backward selection of the parameters was performed (F-in 3.84, F-out 2.71); With a reduction of λ value of Wilks, discrimination between the two groups becomes more prominent. Therefore, after repeating the selection, a set of parameters enabling a best linear discrimination was specified.

In the multivariate stepwise selection discriminant analysis between MIS and CN, 6 parameters shown in Table 2 were selected. The parameters in Table 2 are in the order of inclusion.

TABLE 2
6 parameters used in Melanoma in situ vs. Clark

| Step | Parameter |
|---|---|
| 1 | Horizontal asymmetricity of red component |
| 2 | Deviation of red in rim portion of tumor |
| 3 | Circularity (index of likeness to circle) |
| 4 | Horizontal asymmetricity of green component |
| 5 | Horizontal asymmetricity of shape irrelevant to color |
| 6 | Longitudinal asymmetricity of red component |

In this case, the ultimate λ value of Wilks was 0.69. Sensitivity for melanoma in situ discrimination (percentage of making correct diagnosis on melanoma) was 85.6%, and specificity for melanoma in situ discrimination (percentage of not making incorrect diagnosis of melanoma) was 73.9%. With an adoption of the leave-one-out cross validation method to the percentages, the sensitivity and the specificity for the melanoma in situ discrimination were 69.9% and 85.6%, respectively.

The leave-one-out method is a method wherein, in the case of using N samples, the samples are divided into N−1 practice samples and one evaluation sample to evaluate the evaluation sample by means of a result of learning using the N−1 practice samples, and a percentage of correct evaluation results is calculated for each of N patterns of giving the evaluation sample to use the calculated value as an evaluation value for predictive performance. This method is effective for reducing a bias in data of a small number of samples.

Likewise, 13 parameters shown in Table 3 were used for the multivariate stepwise selection discriminant analysis between TIM and CN. This multivariate stepwise selection discriminant analysis has 15 steps, and the parameters shown in Table 3 are in the order of inclusion.

TABLE 3
13 parameters used in Melanoma vs. Clark

| Step | Parameter |
|---|---|
| 1 | Horizontal asymmetricity of red component |
| 2 | Ratio of black region having luminance of |
| | 100 or less in rim portion of tumor |
| 3 | Longitudinal asymmetricity of shape |
| | irrelevant to color |
| 4 | Average value of blue in overall tumor |
| 5 | Average value of blue in rim portion of tumor |
| 6 | Deviation of blue in overall tumor |
| 7 | Ratio of black region having luminance of |
| | 100 or less in overall tumor |
| 8 | Ratio of black region having luminance of |
| | 100 or less in rim portion of tumor (exclusion) |
| 9 | Average value of green in overall tumor |
| 10 | Deviation of blue in rim portion of tumor |
| 11 | Minimum value of red occupying 0.05% or |
| | more of area of rim portion of tumor |
| 12 | Average value of red in overall tumor |
| 13 | Average value of green in rim portion of tumor |
| 14 | Average value of red in overall tumor |
| 15 | Deviation of red in rim portion of tumor |

(Note)
Parameter of Step 2 is excluded by Step 8

In this case, the ultimate λ value of Wilks was 0.23. Sensitivity for invasive melanoma discrimination and specificity for invasive melanoma discrimination were 94.4% and 98.4%, respectively. With the use of the leave-one-out cross validation method, the sensitivity and the specificity for the invasive melanoma discrimination were 91.7% and 98.4%.

Then, F values for the parameters of the categories of color, texture, asymmetricity, and circularity for discrimination were calculated. Since the size of an F value shows contribution to discrimination, a parameter having a large F value has an important role in discrimination. As a result, the F values were small in the discrimination between MIS and CN (color: 0.0, texture: 26.9, asymmetricity: 37.1, circularity: 7.3), and the F values of the categories of color and texture were large in the discrimination between TIM and CN (color: 189.1, texture: 28.2, asymmetricity: 9.0, circularity: 0.0), thereby indicating that the categories have important roles in discrimination.

<Decision II of Parameters to be Used: Multivariate Discriminant Analysis Using Neural Network System>

In this embodiment, a neural network is used as a discriminator in place of the multivariate stepwise selection discriminant analysis of the foregoing embodiment. Since the neural network is capable of creating a non-linear discrimination borderline which cannot be made by the multivariate stepwise selection discrimination, it is expected to achieve discrimination with higher accuracy. In this embodiment, a neural network obtained by Mackey et al. by expanding a BP (Back Propagation) neural network advantageous for data discrimination and the like through incorporation of statistical technique for the purpose of versatility was used. In practice, the neural network programmed by using MATLAB Rel. 13 (product of The MathWorks, Inc.) was used.

BP is generally formed of three layers, namely, an input layer, an intermediate layer, and an output layer, and the provision of the intermediate layer makes it possible to categorize a problem which cannot be linearly separated by the discriminant analysis. The number of the intermediate layer in this embodiment is 1, and parameters were decided as described below.

The skin images of the cases used in the multivariate stepwise selection discriminant analysis, namely, 23 cases of melanoma in situ (MIS), 36 cases of thin invasive melanoma (TIM), and 188 cases of Clarks nevi (CN), and 56 cases of Reed nevus (RN) were used. With the use of the 62 parameters, the leave-one-out method was performed on each of the parameters, and then the leave-out-one method was performed on the result by adding next parameters. After repeating the same processing by the neural network system, combinations each having a small number of parameters were selected among combinations of parameters capable of achieving diagnosis capability (average of specificity and sensitivity) of at least 85%. As a result, 8 parameters shown in Table 4, which are capable of achieving sensitivity and specificity of invasive melanoma discrimination in the evaluation by the cross validation of 94.8% and 79.0% were selected.

TABLE 4
8 inputs (intermediate layer: 10) selected by neural
network in benign/malignant discrimination of Clark,
Melanoma, Melanoma in situ, and Read nevus

| Step | Parameter |
|---|---|
| 1 | Longitudinal asymmetricity of blue component |
| 2 | Horizontal asymmetricity of shape irrelevant to color |
| 3 | Energy of red in rim portion of tumor |
| 4 | Energy of blue in rim portion of tumor |
| 5 | Ratio of black region having luminance of 100 or |
| | less in rim portion of tumor |
| 6 | Entropy of red in rim portion of tumor |
| 7 | Minimum value of blue in overall tumor |
| 8 | Circularity (index of likeness to circle) |

Comparison Diagnosis Program

The comparison diagnosis program is a program for diagnosing based on plural diagnosis results for pigmentary deposition and compares, for example, diagnosis results for skin images of an identical pigmentary deposition picked up at a different time point to give a diagnosis relating to the pigmentary deposition from the comparison result.

For instance, in Embodiment 1, the melanoma diagnosis program measures the size (area and diameter) of a skin lesion at different time points to store the results in the diagnosis result storage, and then the comparison diagnosis program calculates a degree and a speed of increase in size to determine if the skin lesion is benign or malignant.

In embodiment II, the pigmented macule diagnosis program measures color strength of an identical pigmented macule before and after using a cosmetic agent or drug for diminishing skin pigmentation to store the results in the diagnosis result storage, and then the comparison diagnosis program calculates a degree and a speed of discoloration to examine an effect of the cosmetic agent or the drug.

### INDUSTRIAL APPLICABILITY

According to this invention, it is possible to provide a remote diagnosis apparatus, a remote diagnosis system, a user terminal, a program, and a storage for conveniently diagnosing a pigmentary deposition portion.

1. A remote diagnosis apparatus for diagnosing a skin lesion, which is communicably connected to a first user terminal and a second user terminal provided with a camera device with dermoscope, comprising:
a database for storing data which are received from the first user terminal and relate to the skin lesion, and
a diagnosis program updated based on the data stored in the database and diagnosing skin images for the skin lesion,
the remote diagnosis apparatus receiving a skin image of a pigmentary deposition portion picked up by the camera device with dermoscope from the second user terminal; diagnosing the received skin image for the skin lesion with the use of the diagnosis program; and sending a diagnosis result to a predetermined destination.

2. A remote diagnosis apparatus communicably connected to a first user terminal and a second user terminal provided with a camera device with dermoscope, comprising:
a database for storing data which are received from the first user terminal and relate to a skin lesion;
a diagnosis program which is updated based on the data stored in the database and diagnoses skin images for the skin lesion; and
an image storage for storing the skin images to be diagnosed,
the remote diagnosis apparatus
receiving a skin image of a pigmentary deposition portion picked up by the camera device with dermoscope and source information for identifying the pigmentary deposition portion from the second user terminal and
storing the received skin image in the image storage after associating the source information with the skin image, and
the diagnosis program taking out the skin image stored in the image storage to diagnose the skin image for the skin lesion and sending a diagnosis result to a destination designated by the source information.

3. A remote diagnosis apparatus communicably connected to a first user terminal and a second user terminal provided with a camera device with dermoscope, comprising:
a data receiving unit for receiving data relating to a skin lesion from the first user terminal;
a database for storing data relating to the skin lesion, which stores the received data;
a receiving unit for receiving a skin image of a pigmentary deposition portion picked up by the camera device with dermoscope and source information for identifying the pigmentary deposition portion from the second user terminal;
an image storage for storing the received skin image after associating the source information with the skin image;
a diagnosis program which is updated based on the data stored in the database and takes out the skin image stored in the image storage to diagnose the skin image for the skin lesion; and
a sending unit for sending a diagnosis result of the diagnosis program to a destination designated by the source information.

4. A remote diagnosis apparatus communicably connected to a first user terminal and a second user terminal provided with a camera device with dermoscope, comprising:
a database for storing data which are received from the first user terminal and relate to a skin lesion;
a first diagnosis program which is updated based on the data stored in the database and diagnoses skin images for the skin lesion; and
a second diagnosis program for diagnosing based on a comparison of plural diagnosis results for the skin lesion,
the remote diagnosis apparatus
receiving a first skin image of a pigmentary deposition portion picked up by the camera device with dermoscope from the second user terminal to diagnose the first skin image for the skin lesion with the use of the first diagnosis program,
storing a diagnosis result for the first skin image,
receiving a second skin image of the pigmentary deposition portion same as the first skin image picked up by the camera device with dermoscope at a different time point from the second user terminal and diagnosing the second skin image for the skin lesion with the use of the first diagnosis program,
diagnosing the skin lesion by comparing the diagnosis result for the first skin image with a diagnosis result for the second skin image with the use of the second diagnosis program, and
sending at least one of the diagnosis results of the first diagnosis program and the second diagnosis program to a predetermined destination.

5. A remote diagnosis apparatus communicably connected to a first user terminal and a second user terminal provided with a camera device with dermoscope, comprising:
a database for storing data which are received from the first user terminal and relate to a skin lesion;
a first diagnosis program which is updated based on the data stored in the database and diagnoses skin images for the skin lesion;
a second diagnosis program for diagnosing based on a comparison of plural diagnosis results for the skin lesion;
an image storage for storing the skin images to be diagnosed; and
a diagnosis result storage for storing a diagnosis result,
the remote diagnosis apparatus
receiving a first skin image of a pigmentary deposition portion picked up by the camera device with dermoscope, source information for identifying the pigmentary deposition portion, and first time information relating to a time of sending the first skin image from the second user terminal,
storing the first skin image in the image storage after associating the source information and the first time information with the first skin image,
taking out the first skin image stored in the image storage to diagnose the first skin image for the skin lesion with the use of the first diagnosis program,
storing a diagnosis result for the first skin image in the diagnosis result storage after associating the source information and the first time information with the diagnosis result,
receiving a second skin image of the pigmentary deposition portion same as the first skin image picked up by the camera device with dermoscope at a different time point, source information for identifying the pigmentary deposition portion, and second time information relating to a time of sending the second skin image from the second user terminal,
storing the second skin image in the image storage after associating the source information and the second time information with the second skin image,
taking out the second skin image stored in the image storage to diagnose the second skin image for the skin lesion with the use of the first diagnosis program,
comparing the diagnosis result for the first skin image with a diagnosis result for the second skin image with the use of the second diagnosis program to diagnose further for the skin lesion, and
sending at least one of the diagnosis results of the first diagnosis program and the second diagnosis program to a destination designated by the source information.

6. A remote diagnosis apparatus communicably connected to a first user terminal and a second user terminal provided with a camera device with dermoscope, comprising:
a database for storing data relating to a skin lesion;
a data receiving unit for receiving data relating to the skin lesion from the first user terminal;
a database for storing data relating to the skin lesion, which stores the received data;
a receiving unit for receiving a skin image of a pigmentary deposition portion picked up by the camera device with dermoscope, source information for identifying the pigmentary deposition portion, and time information relating to a time of sending the skin image from the second user terminal;
an image storage for storing the received skin image after associating the source information and the time information with the skin image;
a first diagnosis program which is updated based on the data stored in the database and takes out the skin image stored in the image storage to diagnose the skin image for the skin lesion;
a diagnosis result storage for storing a diagnosis result after associating the source information and the time information with the diagnosis result;
a second diagnosis program capable of diagnosing the skin lesion based on plural diagnosis results having different time information; and
a sending unit for sending at least one of diagnosis results of the first diagnosis program and the second diagnosis program to a destination designated by the source information.

7. The remote diagnosis apparatus according to claim 1, characterized in that the second user terminal is provided with a portable telephone function.

8. The remote diagnosis apparatus according to claim 1, characterized in that the second user terminal communicates via the Internet.

9. A remote diagnosis apparatus communicably connected to
a database control server provided with a database for storing data relating to a skin lesion and
a user terminal provided with a camera device with dermoscope, comprising
a diagnosis program which is updated based on the data stored in the database and diagnoses skin images for the skin lesion,
the remote diagnosis apparatus
receiving a skin image of a pigmentary deposition portion picked up by the camera device with dermoscope from the user terminal,
diagnosing the received skin image for the skin lesion with the use of the diagnosis program, and
sending a diagnosis result to a predetermined destination.

10. A remote diagnosis apparatus communicably connected to
a database control server provided with a database for storing data relating to a skin lesion and
a user terminal provided with a camera device with dermoscope, comprising
a diagnosis program which is updated based on the data stored in the database and diagnoses skin images for the skin lesion and
an image storage for storing the skin images to be diagnosed,
the remote diagnosis apparatus
receiving a skin image of a pigmentary deposition portion picked up by the camera device with dermoscope and source information for identifying the pigmentary deposition portion from the user terminal,
storing the received skin image in the image storage after associating the source information with the skin image,
diagnosing the skin image stored in the image storage for the skin lesion with the use of the diagnosis program, and
sending a diagnosis result to a destination designated by the source information.

11. A remote diagnosis apparatus communicably connected to
a database control server provided with a database for storing data relating to a skin lesion and
a user terminal provided with a camera device with dermoscope, comprising:
a receiving unit for receiving a skin image of a pigmentary deposition portion picked up by the camera device with dermoscope and source information for identifying the pigmentary deposition portion from the user terminal;
an image storage for storing the received skin image after associating the source information with the skin image;
a diagnosis program which is updated based on the data stored in the database and takes out the skin image stored in the image storage to diagnose the skin image for the skin lesion; and
a sending unit for sending a diagnosis result of the diagnosis program to a destination designated by the source information.

12. A remote diagnosis apparatus communicably connected to
a database control server provided with a database for storing data relating to a skin lesion and
a user terminal provided with a camera device with dermoscope, comprising
a first diagnosis program which is updated based on the data stored in the database and diagnoses skin images for the skin lesion and
a second diagnosis program for diagnosing based on plural diagnosis results for the skin lesion,
the remote diagnosis apparatus
receiving a first skin image of a pigmentary deposition portion picked up by the camera device with dermoscope from the user terminal,
diagnosing the first skin image for the skin lesion with the use of the first diagnosis program,
storing a diagnosis result for the first skin image,
receiving a second skin image of the pigmentary deposition portion same as the first skin image picked up by the camera device with dermoscope at a different time point from the user terminal,
diagnosing the second skin image for the skin lesion with the use of the first diagnosis program,
comparing the diagnosis result for the first skin image with a diagnosis result for the second skin image with the use of the second diagnosis program, and
sending at least one of the diagnosis results of the first diagnosis program and the second diagnosis program to a destination designated by the source information.

13. A remote diagnosis apparatus communicably connected to
a database control server provided with a database for storing data relating to a skin lesion and
a user terminal provided with a camera device with dermoscope, comprising:
a first diagnosis program which is updated based on the data stored in the database and diagnoses skin images for the skin lesion;
a second diagnosis program for diagnosing based on plural diagnosis results for the skin lesion;
an image storage for storing the skin images to be diagnosed; and
a diagnosis result storage for storing a diagnosis result,
the remote diagnosis apparatus
receiving a first skin image of a pigmentary deposition portion picked up by the camera device with dermoscope, source information for identifying the pigmentary deposition portion, and first time information relating to a time of sending the first skin image from the user terminal,
storing the first skin image in the image storage after associating the source information and the first time information with the first skin image,
taking out the first skin image stored in the image storage to diagnose the first skin image for the skin lesion with the use of the first diagnosis program,
storing a diagnosis result for the first skin image in the diagnosis result storage after associating the source information and the first time information with the diagnosis result,
receiving a second skin image of the pigmentary deposition portion same as the first skin image picked up by the camera device with dermoscope at a different time point, source information for identifying the pigmentary deposition portion, and second time information relating to a time of sending the second skin image from the user terminal,
storing the second skin image in the diagnosis result storage after associating the source information with the second skin image,
taking out the stored second skin image to diagnose the second skin image for the skin lesion with the use of the first diagnosis program,
diagnosing through a comparison between the diagnosis result for the first skin image and a diagnosis result for the second skin image with the use of the second diagnosis program, and
sending at least one of the diagnosis results of the first diagnosis program and the second diagnosis program to a destination designated by the source information.

14. A remote diagnosis apparatus communicably connected to
a database control server provided with a database for storing data relating to a skin lesion and
a user terminal provided with a camera device with dermoscope, comprising:
a receiving unit for receiving a skin image of a pigmentary deposition portion picked up by the camera device with dermoscope, source information for identifying the pigmentary deposition portion, and time information relating to a time of sending the skin image from the user terminal;
an image storage for storing the received skin image after associating the source information and the time information with the skin image;
a first diagnosis program which is updated based on the data stored in the database and takes out the skin image stored in the image storage to diagnose the skin image for a skin lesion;
a diagnosis result storage for storing a diagnosis result for the skin image after associating the source information and the time information with the diagnosis result;
a second diagnosis program capable of diagnosing the skin lesion based on plural diagnosis results for the skin lesion of the skin image, which are stored in the diagnosis result storage and different in time information; and
a sending unit for sending at least one of diagnosis results of the first diagnosis program and the second diagnosis program to a destination designated by the source information.

15. A remote diagnosis apparatus communicably connected to a user terminal provided with a camera device with dermoscope, comprising
receiving a skin image of a pigmentary deposition portion picked up by the camera device with dermoscope;
diagnosing the received skin image for the skin lesion; and
sending a diagnosis result to a predetermined destination.

16. A remote diagnosis apparatus communicably connected to a user terminal provided with a camera device with dermoscope, comprising:
a diagnosis program for diagnosing skin images for the skin lesion and an image storage for storing the skin images to be diagnosed;
the remote diagnosis apparatus receiving a skin image of a pigmentary deposition portion picked up by the camera device with dermoscope and source information for identifying the pigmentary deposition portion,
storing the received skin image in the image storage after associating the source information with the skin image,
taking out the skin image stored in the image storage and diagnosing the skin image for the skin lesion with the use of the diagnosis program, and
sending a diagnosis result to a destination designated by the source information.

17. A remote diagnosis apparatus communicably connected to a user terminal provided with a camera device with dermoscope, comprising:
a receiving unit for receiving a skin image of a pigmentary deposition portion picked up by the camera device with dermoscope and source information for identifying the pigmentary deposition portion;
an image storage for storing the received skin image after associating the source information with the skin image;
a diagnosis program for taking out the skin image stored in the image storage to diagnose the skin image for the skin lesion; and
a sending unit for sending a diagnosis result of the diagnosis program to a destination designated by the source information.

18. The remote diagnosis apparatus according to claim 9, characterized in that the user terminal is provided with a portable telephone function.

19. The remote diagnosis apparatus according to claim 9, characterized in that the user terminal communicates via the Internet.

20. The remote diagnosis apparatus according to claim 1, characterized in that the dermoscope comprises a polarizing filter.

21. The remote diagnosis apparatus according to claim 1, characterized in that the skin lesion of a diagnosis object is melanoma.

22. A remote diagnosis system for diagnosing a skin lesion, comprising:
a first user terminal;
a second user terminal provided with a camera device with dermoscope;
a remote diagnosis apparatus communicably connected to the first user terminal and the second user terminal and comprises a database for storing data which are received from the first user terminal and relate to the skin lesion; and a diagnosis program relating to the skin lesion which is updated based on the data stored in the database, wherein
the second user terminal sends a skin image of a pigmentary deposition portion picked up by the camera device with dermoscope to the remote diagnosis apparatus, and
the remote diagnosis apparatus
receives the skin image,
diagnoses the received skin image for the skin lesion with the use of the diagnosis program, and
sends a diagnosis result to a predetermined destination.

23. A remote diagnosis system for diagnosing a skin lesion, comprising:
a first user terminal;
a second user terminal provided with a camera device with dermoscope; and
a remote diagnosis apparatus communicably connected to the first and the second user terminals, wherein
the first user terminal comprises a sending unit for sending data relating to the skin lesion to the remote diagnosis apparatus,
the second user terminal comprises a sending unit for sending a skin image of a pigmentary deposition portion picked up by the camera device with dermoscope to the remote diagnosis apparatus, and
the remote diagnosis apparatus comprises
a data receiving unit for receiving the data from the first user terminal,
a database for storing the data which are received from the first user terminal and relate to the skin lesion,
an image receiving unit for receiving the skin image from the second user terminal,
a diagnosis program which is updated based on the data stored in the database and diagnoses the skin image received from the second user terminal for the skin lesion, and
a sending unit for sending a diagnosis result to a predetermined destination.

24. A remote diagnosis system for diagnosing a skin lesion, comprising:
a first user terminal;
a database control server communicably connected to the first user terminal and provided with a database for storing data which are received from the first user terminal and relate to the skin lesion;
a second user terminal provided with a camera device with dermoscope; and
a remote diagnosis apparatus communicably connected to the database control server and the second user terminal and provided with a diagnosis program which is updated based on the data stored in the database and diagnoses skin images for the skin lesion, wherein
the second user terminal sends a skin image of a pigmentary deposition portion picked up by the camera device with dermoscope; and
the remote diagnosis apparatus
diagnoses the skin image received from the second user terminal for the skin lesion with the use of the diagnosis program and
sends a diagnosis result to a predetermined destination.

25. A remote diagnosis system for diagnosing a skin lesion, comprising:
a first user terminal;
a database control server communicably connected to the first user terminal;
a second user terminal provided with a camera device with dermoscope; and
a remote diagnosis apparatus communicably connected to the database control server and the second user terminal, wherein
the first user terminal comprises a sending unit for sending data relating to the skin lesion to the remote diagnosis apparatus,
the database control server comprises
a data receiving unit for receiving the data from the first user terminal and
a database for storing the data which are received from the first user terminal and relate to the skin lesion;
the second user terminal comprises a sending unit for sending a skin image of a pigmentary deposition portion picked up by the camera device with dermoscope to the remote diagnosis apparatus, and
the remote diagnosis apparatus comprises
a receiving unit for receiving the skin image from the second user terminal;
a diagnosis program which is updated based on the data stored in the database and diagnoses the skin image received from the second user terminal for the skin lesion; and
a sending unit for sending a diagnosis result to a predetermined destination.

26. A remote diagnosis system for diagnosing a skin lesion, comprising:
a first user terminal;
a second user terminal provided with a camera device with dermoscope; and
a remote diagnosis apparatus communicably connected to the first and the second user terminals and comprises a database for storing data which are received from the first user terminal and relate to the skin lesion, a first diagnosis program which is updated based on the data stored in the database and diagnoses skin images for the skin lesion, and a second diagnosis program for diagnosing based on plural diagnosis results for the skin lesion, wherein
the second user terminal sends a first skin image of a pigmentary deposition portion picked up by the camera device with dermoscope to the remote diagnosis apparatus;
the remote diagnosis apparatus
receives the first skin image and
diagnoses the received skin image for the skin lesion with the use of the first diagnosis program;
the second user terminal sends a second skin image of the pigmentary deposition portion same as the first skin image picked up by the camera device with dermoscope at a different time point to the remote diagnosis apparatus;
the remote diagnosis apparatus
receives the second skin image and
diagnoses the received second skin image for the skin lesion with the use of the first diagnosis program;
the remote diagnosis apparatus
diagnoses the skin lesion by comparing the diagnosis result for the first skin image with a diagnosis result for the second skin image with the use of the second diagnosis program and
sends at least one of the diagnosis results of the first diagnosis program and the second diagnosis program to a predetermined destination.

27. A remote diagnosis system for diagnosing a skin lesion, comprising:
a first user terminal;
a second user terminal provided with a camera device with dermoscope; and
a remote diagnosis apparatus communicably connected to the first and the second user terminals, wherein
the first user terminal comprises
a sending unit for sending data relating to the skin lesion to the remote diagnosis apparatus;
the second user terminal comprises a sending unit for sending a skin image of a pigmentary deposition portion picked up by the camera device with dermoscope, source information for identifying the pigmentary deposition portion, and time information relating to a time of sending the skin image to the remote diagnosis apparatus; and
the remote diagnosis apparatus comprises
a data receiving unit for receiving the data from the first user terminal;
a database for storing the data which are received from the first user terminal and relate to the skin lesion;
an image receiving unit for receiving the skin image, the source information, and the time information from the second user terminal;
an image storage for storing the received skin image after associating the source information and the time information with the skin image;
a first diagnosis program which is updated based on the data stored in the database and diagnoses the skin image taken out from the storage for the skin lesion;
a second diagnosis program capable of diagnosing the skin lesion based on plural diagnosis results for the skin lesion of the skin image, which are stored in the diagnosis result storage and different in time information; and
a sending unit for sending at least one of diagnosis results of the first diagnosis program and the second diagnosis program to a predetermined destination.

28. A remote diagnosis system for diagnosing a skin lesion, comprising:
a first user terminal;
a database control server communicably connected to the first user terminal and provided with a database for storing data which are received from the first user terminal and relate to the skin lesion;
a second user terminal provided with a camera device with dermoscope; and
a remote diagnosis apparatus communicably connected to the second user terminal and comprising a first diagnosis program which is updated based on the data stored in the database and diagnoses skin images for the skin lesion, and a second diagnosis program for diagnosing the skin lesion based on a comparison of plural diagnosis results, wherein
the second user terminal sends a first skin image of a pigmentary deposition portion picked up by the camera device with dermoscope to the remote diagnosis apparatus;
the remote diagnosis apparatus
receives the first skin image and
diagnoses the received first skin image for the skin lesion with the use of the first diagnosis program;
the second user terminal sends a second skin image of the pigmentary deposition portion same as the first skin image picked up by the camera device with dermoscope to the remote diagnosis apparatus;
the remote diagnosis apparatus
receives the second skin image,
diagnoses the received second skin image for the skin lesion with the use of the first diagnosis program,
diagnoses the skin lesion based on a comparison between the diagnosis result for the first skin image and a diagnosis result for the second skin image with the use of the second diagnosis program, and
sends at least one of the diagnosis results of the first diagnosis program and the second diagnosis program to the second user terminal.

29. A remote diagnosis system for diagnosing a skin lesion, comprising:
a first user terminal;
a database control server communicably connected to the first user terminal;
a second user terminal provided with a camera device with dermoscope; and
a remote diagnosis apparatus communicably connected to the database control server and the second user terminal, wherein
the first user terminal comprises a sending unit for sending data relating to the skin lesion to the remote diagnosis apparatus;
the database control server comprises
a data receiving unit for receiving the data from the first user terminal and
a database for storing the data received from the first user terminal;
the second user terminal comprises a sending unit for sending a skin image of a pigmentary deposition portion picked up by the camera device with dermoscope, source information for identifying the pigmentary deposition portion, and time information relating to a time of sending the skin image to the remote diagnosis apparatus; and
the remote diagnosis apparatus comprises
an image receiving unit for receiving the skin image, the source information, and the time information from the second user terminal;
an image storage for storing the skin image after associating the source information and the time information with the skin image;
a first diagnosis program which is updated based on the data stored in the database and diagnoses the received skin image for the skin lesion;
a second diagnosis program capable of diagnosing based on plural diagnosis results for the skin lesion;
a diagnosis result storage for storing the diagnosis results; and
a sending unit for sending the diagnosis result to a predetermined destination.

30. The system according to claim 22, characterized in that the second user terminal is provided with a portable telephone function.

31. The system according to claim 22, characterized in that the second user terminal communicates via the Internet.

32. A remote diagnosis system for diagnosing a skin lesion, comprising:
a user terminal provided with a camera device with dermoscope and
a remote diagnosis apparatus communicably connected to the user terminal, wherein
the user terminal sends a skin image of a pigmentary deposition portion picked up by the camera device with dermoscope and source information for identifying the pigmentary deposition portion to the remote diagnosis apparatus, and
the remote diagnosis apparatus diagnoses the skin image received from the user terminal for the skin lesion to send a diagnosis result to a destination designated by the source information.

33. A remote medical diagnosis system for diagnosing presence/absence and a disease state of a skin lesion, comprising:
a user terminal provided with a camera device with dermoscope and
a remote diagnosis apparatus communicably connected to the user terminal, wherein
the user terminal comprises a sending unit for sending a skin image of a pigmentary deposition portion picked up by the camera device with dermoscope to the remote diagnosis apparatus, and
the remote diagnosis apparatus comprises
a receiving unit for receiving the skin image of the pigmentary deposition portion picked up by the camera device with dermoscope and source information for identifying the pigmentary deposition portion;
an image storage for storing the received skin image after associating the source information with the skin image;
a diagnosis program taking out the skin image from the image storage to diagnose the skin image for the skin lesion; and
a sending unit for sending a diagnosis result obtained by the diagnosis to a destination designated by the source information.

34. A remote diagnosis system for diagnosing a skin lesion, comprising:
a user terminal provided with a camera device with dermoscope;
a remote diagnosis apparatus communicably connected to the user terminal;
a first diagnosis program diagnosing skin images for the skin lesion;
and a second diagnosis program for diagnosing based on plural diagnosis results for the skin lesion, wherein
the user terminal sends a first skin image of a pigmentary deposition portion picked up by the camera device with dermoscope to the remote diagnosis apparatus;
the remote diagnosis apparatus
receives the first skin image and
diagnoses the received first skin image for the skin lesion with the use of the first diagnosis program, and
the user terminal sends a second skin image of the pigmentary deposition portion same as the first skin image picked up by the camera device with dermoscope to the remote diagnosis apparatus, and
the remote diagnosis apparatus
receives the second skin image,
diagnoses the second skin image for the skin lesion with the use of the first diagnosis program,
diagnoses the skin lesion based on a comparison between the diagnosis result for the first skin image and a diagnosis result for the second skin image with the use of the second diagnosis program, and
sends at least one of the diagnosis results of the first diagnosis program and the second diagnosis program to a destination designated by the source information.

35. A remote medical diagnosis system for diagnosing presence/absence and a disease state of a skin lesion, comprising:
a user terminal provided with a camera device with dermoscope and
a remote diagnosis apparatus communicably connected to the user terminal, wherein
the remote diagnosis apparatus comprises:
an image receiving unit for receiving a skin image of a pigmentary deposition portion picked up by the camera device with dermoscope, source information for identifying the pigmentary deposition portion, and time information relating to a time of sending the skin image;
an image storage for storing the received skin image after associating the source information and the time information with the skin image;
a first diagnosis program for taking out the skin image from the image storage to diagnose the skin image for the skin lesion;
a diagnosis result storage for storing a diagnosis result after associating the source information and the time information with the diagnosis result;
a second diagnosis program for diagnosing the skin lesion based on plural diagnosis results for the skin lesion having different time information; and
a sending unit for sending at least one of diagnosis results of the first diagnosis program and the second diagnosis program to a destination designated by the source information.

36. The system according to claim 32, characterized in that the user terminal is provided with a portable telephone function.

37. The system according to claim 32, characterized in that the user terminal communicates via the Internet.

38. The system according to claim 22, characterized in that the dermoscope comprises a polarizing filter.

39. The system according to claim 22, characterized in that the skin lesion which is a subject of the diagnosis is melanoma.

40. A user terminal comprising:
a camera device with dermoscope provided with a polarizing filter;
a sending unit capable of sending a skin image of a pigmentary deposition portion picked up by the camera device with dermoscope and source information for identifying the pigmentary deposition portion;
a receiving unit capable of receiving a diagnosis result obtained by diagnosing the skin image for a skin lesion or a pigmentary deposition; and
a portable telephone function.

41. A user terminal characterized in that the sending/receiving unit sends/receives to/from any one of the remote diagnosis apparatuses defined in claim 1.

42. The user terminal according to claim 40, characterized in that the sending unit capable of further sending time information relating to a time of sending.

43. The user terminal according to claim 40, characterized by communicating with the remote diagnosis apparatus via the Internet.

44. The user terminal according to claim 40, characterized in that the skin lesion which is a subject of the diagnosis is melanoma.

45. A program for causing a computer to execute a remote diagnosis for diagnosing a skin lesion, the computer being communicably connected to a first user terminal and a second user terminal provided with a camera device with dermoscope and comprising a database for storing data which are received from the first user terminal and relate to the skin lesion, a diagnosis program which is updated based on the data stored in the database and diagnoses skin images for the skin lesion, and an image storage for storing the skin images to be diagnosed, wherein
the program causes the computer to execute
a step for receiving a skin image of a pigmentary deposition portion picked up by the camera device with dermoscope and source information for identifying the pigmentary deposition portion from the second user terminal;
a step for storing the received skin image in the image storage after associating the source information with the skin image;
a step for taking out the skin image stored in the image storage and diagnosing the skin image for the skin lesion; and
a step for sending a diagnosis result to a destination designated by the source information.

46. A program for causing a computer to execute a remote diagnosis for diagnosing presence/absence and a disease state of a skin lesion, the computer being communicably connected to a first user terminal and a second user terminal provided with a camera device with dermoscope and comprising a database for storing data which are received from the first user terminal and relate to the skin lesion, a first diagnosis program which is updated based on the data stored in the database and diagnoses skin images for the skin lesion, a second diagnosis program for diagnosing the skin lesion based on a comparison of plural diagnosis results for the skin lesion, an image storage for storing the skin images to be diagnosed, and a diagnosis result storage for storing a diagnosis result, wherein
the program causes the computer to execute a step for receiving a first skin image of a pigmentary deposition portion picked up by the camera device with dermoscope, source information for identifying the pigmentary deposition portion, and time information relating to a time of sending the skin image from the second user terminal,
a step for storing the first skin image in the image storage after associating the source information and the time information with the first skin image;
a step for taking out the first skin image stored in the image storage to diagnose the first skin image for the skin lesion with the use of the first diagnosis program;
a step for storing a diagnosis result for the first skin image in the diagnosis result storage after associating the source information and the time information with the diagnosis result;
a step for receiving a second skin image of the pigmentary deposition portion same as the first skin image picked up by the camera device with dermoscope at a different time point, source information for identifying the pigmentary deposition portion, and time information relating to a time of sending the skin image from the second user terminal,
a step for storing the second skin image in the image storage after associating the source information and the time information with the second skin image;
a step for taking out the second skin image stored in the image storage to diagnose the second skin image for the skin lesion with the use of the first diagnosis program;
a step for diagnosing the skin lesion based on a comparison between the diagnosis result for the first skin image and a diagnosis result for the second skin image with the use of the second diagnosis program; and
a step for sending at least one of the diagnosis results of the first diagnosis program and the second diagnosis program to a destination designated by the source information.

47. A program for causing a computer to execute a remote diagnosis for diagnosing a skin lesion,
the computer being communicably connected to a database control server communicably connected to the first user terminal and provided with a database for storing data which are received from the first user terminal and relate to the skin lesion and
a second user terminal provided with a camera device with dermoscope
and comprising a diagnosis program which is updated based on the data stored in the database and diagnoses skin images for the skin lesion and an image storage for storing the skin images to be diagnosed, wherein
the program causes the computer to execute a step for receiving a skin image of a pigmentary deposition portion picked up by the camera device with dermoscope and source information for identifying the pigmentary deposition portion from the second user terminal;
a step for storing the received skin image in the image storage after associating the source information with the skin image;
a step for taking out the stored skin image and diagnosing the skin image for the skin lesion with the use of the diagnosis program; and
a step for sending a diagnosis result to a destination designated by the source information.

48. A program for causing a computer to execute a remote diagnosis for diagnosing a skin lesion, the computer being communicably connected to
a database control server communicably connected to the first user terminal and provided with a database for storing data which are received from the first user terminal and relate to the skin lesion and
a second user terminal provided with a camera device with dermoscope
and comprising
a first diagnosis program which is updated based on the data stored in the database and diagnoses skin images for the skin lesion;
a second diagnosis program for diagnosing the skin lesion based on a comparison of plural diagnosis results for the skin lesion;
an image storage for storing the skin images to be diagnosed; and
a diagnosis result storage for storing a diagnosis result, wherein
the program causes the computer to execute
a step for receiving a first skin image of a pigmentary deposition portion picked up by the camera device with dermoscope, source information for identifying the pigmentary deposition portion, and time information relating to a time of sending the skin image from the second user terminal;
a step for storing the received first skin image in the image storage after associating the source information and the time information with the first skin image;
a step for taking out the stored first skin image to diagnose the first skin image for the skin lesion with the use of the first diagnosis program;
a step for storing a diagnosis result for the first skin image in the diagnosis result storage after associating the source information and the time information with the diagnosis result;
a step for receiving a second skin image of the pigmentary deposition portion same as the first skin image picked up by the camera device with dermoscope, source information for identifying the skin lesion, and time information relating to a time of sending the skin image from the second user terminal;
a step for storing the received second skin image after associating the source information and the time information with the second skin image;
a step for taking out the stored second skin image to diagnose the second skin image for the skin lesion with the use of the first diagnosis program;
a step for diagnosing the skin lesion based on a comparison between the diagnosis result for the first skin image and a diagnosis result for the second skin image with the use of the second diagnosis program; and
a step for sending at least one of the diagnosis results of the first diagnosis program and the second diagnosis program to a destination designated by the source information.

49. A program for causing a computer to execute a remote diagnosis for diagnosing a skin lesion,
the computer being communicably connected to
a user terminal provided with a camera device with dermoscope and comprising
a diagnosis program for diagnosing skin images for the skin lesion and
an image storage for storing the skin images to be diagnosed, wherein
the program causes the computer to execute a step for receiving a skin image picked up by the camera device with dermoscope and source information for identifying the user terminal with the dermoscope;
a step for storing the received skin image in the image storage after associating the source information with the skin image;
a step for taking out the skin image stored in the image storage and diagnosing the skin image for the skin lesion with the use of the diagnosis program; and
a step for sending a diagnosis result to a destination designated by the source information.

50. A program for causing a computer to execute a remote diagnosis for diagnosing a skin lesion,
the computer being communicably connected to
a user terminal provided with a camera device with dermoscope and comprising:
a first diagnosis program for diagnosing the skin images for the skin lesion;
a second diagnosis program for diagnosing the skin lesion based on a comparison of plural diagnosis results for the skin lesion;
an image storage for storing the skin images to be diagnosed; and
a diagnosis result storage for storing a diagnosis result, wherein
the program causes the computer to execute a step for receiving a first skin image of a pigmentary deposition portion picked up by the camera device with dermoscope, source information for identifying the pigmentary deposition portion, and time information relating to a time of sending;
a step for storing the first skin image in the image storage after associating the source information and the time information with the first skin image;
a step for taking out the first skin image stored in the image storage to diagnose the first skin image for the skin lesion with the use of the first diagnosis program;
a step for storing a diagnosis result for the first skin image in the diagnosis result storage after associating the source information and the time information with the diagnosis result;
a step for receiving a second skin image of the pigmentary deposition portion same as the first skin image picked up by the camera device with dermoscope, source information for identifying the second pigmentary deposition portion, and time information relating to a time of sending;
a step for storing the received second skin image in the image storage after associating the source information and the time information with the second skin image;
a step for taking out the second skin image stored in the image storage to diagnose the second skin image for the skin lesion with the use of the first diagnosis program;
a step for diagnosing the skin lesion based on a comparison between the diagnosis result for the first skin image and a diagnosis result for the second skin image with the use of the second diagnosis program; and
a step for sending at least one of the diagnosis results of the first diagnosis program and the second diagnosis program to a destination designated by the source information.

51. A storage storing the program defined in claim 45 in a machine-readable form.

52. A remote diagnosis apparatus communicably connected to a user terminal provided with a camera device with dermoscope, the remote diagnosis apparatus comprising:
a first diagnosis program diagnosing skin images for the pigmentary deposition and
a second diagnosis program for diagnosing based on plural diagnosis results for the pigmentary deposition and
receiving a first skin image of a pigmentary deposition portion picked up by the camera device with dermoscope from the user terminal;
diagnosing the first skin image for the pigmentary deposition with the use of the first diagnosis program;
storing a diagnosis result for the first skin image;
receiving a second skin image of the pigmentary deposition portion same as the first skin image picked up by the camera device with dermoscope at a different time point from the user terminal;
diagnosing the second skin image for the pigmentary deposition with the use of the first diagnosis program;
comparing the diagnosis result for the first skin image with a diagnosis result for the second skin image with the use of the second diagnosis program; and
sending at least one of the diagnosis results of the first diagnosis program and the second diagnosis program to a destination designated by the source information.

53. A remote diagnosis apparatus communicably connected to a user terminal provided with a camera device with dermoscope, the remote diagnosis apparatus comprising:
a first diagnosis program diagnosing skin images for the pigmentary deposition;
a second diagnosis program for diagnosing based on plural diagnosis results for the pigmentary deposition;
an image storage for storing the skin images to be diagnosed; and
a diagnosis result storage for storing a diagnosis result,
receiving a first skin image of a pigmentary deposition portion picked up by the camera device with dermoscope, source information for identifying the pigmentary deposition portion, and first time information relating to a time of sending the first skin image from the user terminal,
storing the received first skin image in the image storage after associating the source information and the first time information with the first skin image,
taking out the first skin image stored in the image storage to diagnose the first skin image for the pigmentary deposition with the use of the first diagnosis program,
storing a diagnosis result for the first skin image in the diagnosis result storage after associating the source information and the first time information with the diagnosis result,
receiving a second skin image of the pigmentary deposition portion same as the first skin image picked up by the camera device with dermoscope at a different time point, source information for identifying the pigmentary deposition portion, and second time information relating to a time of sending from the user terminal, storing the second skin image in the diagnosis result storage after associating the source information with the second skin image,
taking out the stored second skin image to diagnose the second skin image for the pigmentary deposition with the use of the first diagnosis program,
diagnosing through a comparison between the diagnosis result for the first skin image and a diagnosis result for the second skin image with the use of the second diagnosis program, and sending at least one of the diagnosis results of the first diagnosis program and the second diagnosis program to a destination designated by the source information.

54. A remote diagnosis apparatus communicably connected to a user terminal provided with a camera device with dermoscope, the remote diagnosis apparatus comprising:
a receiving unit for receiving a skin image of a pigmentary deposition portion picked up by the camera device with dermoscope, source information for identifying the pigmentary deposition portion, and time information relating to a time of sending the skin image from the user terminal;
an image storage for storing the received skin image after associating the source information and the time information with the skin image;
a first diagnosis program for taking out the skin image stored in the image storage to diagnose the skin image for a pigmentary deposition;
a diagnosis result storage for storing a diagnosis result after associating the source information and the time information with the diagnosis result for the skin image;
a second diagnosis program capable of diagnosing the pigmentary deposition based on plural diagnosis results for the pigmentary deposition of the skin image, which are stored in the diagnosis result storage and different in time information; and
a sending unit for sending at least one of diagnosis results of the first diagnosis program and the second diagnosis program to a destination designated by the source information.

55. The remote diagnosis apparatus according to claim 52, characterized in that the user terminal is provided with a portable telephone function.

56. The remote diagnosis apparatus according to claim 52, characterized in that the user terminal communicates via the Internet.

57. A remote diagnosis system for diagnosing a skin having a pigmentary deposition, comprising
a user terminal provided with a camera device with dermoscope and
a remote diagnosis apparatus communicably connected to the user terminal and comprising
a first diagnosis program for diagnosing skin images for the pigmentary deposition and
a second diagnosis program capable of diagnosing the pigmentary deposition based on a comparison of plural results of the diagnosis, wherein
the user terminal sends a first skin image of a pigmentary deposition portion picked up by the camera device with dermoscope to the remote diagnosis apparatus;
the remote diagnosis apparatus
receives the first skin image and
diagnoses the received first skin image for the pigmentary deposition with the use of the first diagnosis program;
the user terminal sends a second skin image of the pigmentary deposition portion same as the first skin image picked up by the camera device with dermoscope to the remote diagnosis apparatus;
the remote diagnosis apparatus
receives the second skin image;
diagnoses the received second skin image for the pigmentary deposition with the use of the first diagnosis program;
diagnosing the pigmentary deposition based on a comparison between the diagnosis result for the first skin image with a diagnosis result for the second skin image with the use of the second diagnosis program; and
sends at least one of the diagnosis results of the first diagnosis program and the second diagnosis program to the second user terminal.

58. A remote diagnosis system for diagnosing a pigmentary deposition, comprising:
a user terminal provided with a camera device with dermoscope and
a remote diagnosis apparatus communicably connected to the user terminal, wherein
the user terminal comprises a sending unit for sending a skin image of a pigmentary deposition portion picked up by the camera device with dermoscope, source information for identifying the pigmentary deposition portion, and time information relating to a time of sending the skin image to the remote diagnosis apparatus, and
the remote diagnosis apparatus comprises:
an image receiving unit for receiving the skin image, the source information, and the time information from the second user terminal;
an image storage for storing the received skin image after associating the source information and the time information with the skin image;
a first diagnosis program for diagnosing the received skin image for the pigmentary deposition;
a second diagnosis program capable of diagnosing based on plural diagnosis results for the pigmentary deposition;
a diagnosis result storage for storing the diagnosis result; and
a sending unit for sending the diagnosis result to a predetermined destination.

59. The system according to claim 57, characterized in that the user terminal is provided with a portable telephone function.

60. The system according to claim 57, characterized in that the user terminal communicates via the Internet.

61. A remote diagnosis method for diagnosing a pigmentary deposition other than skin lesions using a remote diagnosis apparatus which is communicably connected to a user terminal provided with a camera device with dermoscope and comprises:
an image receiving unit for receiving a skin image to be diagnosed from the user terminal;
an image storage for storing a skin image;
a first diagnosis program for diagnosing skin images for the pigmentary deposition;
a second diagnosis program diagnosing based on plural diagnosis results for the pigmentary deposition;
a diagnosis result storage for storing the diagnosis results; and
a diagnosis result sending unit for sending at least one of the diagnosis results of the first diagnosis program and the second diagnosis program to the user terminal, wherein
the image receiving unit receives a first skin image of a pigmentary deposition portion picked up by the camera device with dermoscope, source information for identifying the pigmentary deposition portion, and first time information relating to a time of sending the first skin image from the user terminal;
the image storage stores the first skin image after associating the source information and the first time information with the skin image;
the first diagnosis program takes out the first skin image stored in the image storage to diagnose the first skin image for the pigmentary deposition;
the diagnosis result storage stores a diagnosis result for the first skin image after associating the source information and the first time information with the diagnosis result;
the image receiving unit receives a second skin image of the pigmentary deposition portion same as the first skin image picked up by the camera device with dermoscope at a different time point, source information for identifying the pigmentary deposition portion, and second time information relating to a sending time from the user terminal;
the diagnosis result storage stores the received second skin image after associating the source information with the second skin image;
the first diagnosis program takes out the stored second skin image to diagnose the second skin image for the pigmentary deposition;
the second diagnosis program diagnoses through a comparison between the diagnosis result for the first skin image and the diagnosis result for the second skin image;
the diagnosis result sending unit sends at least one of the diagnosis results of the first diagnosis program and the second diagnosis program to a destination designated by the source information.

62. A diagnosis program which is updated based on plural skin images stored in a database of which diagnoses have been determined and used for diagnosing skin images to be diagnosed for a skin lesion, the diagnosis program causing a computer to execute
a step for performing image processing by separating a pigmentary disorder portion of a skin lesion from a peripheral normal portion in each of plural skin images on which the diagnoses have been made and separating a rim portion of the separated pigmentary disorder portion;
a step for deciding parameters to be used for discrimination of characteristics of the pigmentary disorder, such as a color, a texture, an asymmetricity, and a circularity; and
a step for performing evaluation of each of the parameters using a predetermined system on the diagnosed skin images with the use of each of the parameters, performing evaluation of each of other parameters using the predetermined system on the diagnosed skin images with the use of each of other parameters, repeating the same operation until a diagnosis capability of any one of parameter combinations reaches a predetermined value, and, ultimately, selecting a parameter combination having a smallest number of parameters from the parameter combinations having the diagnosis capability equal to or more than the predetermined value.

63. The diagnosis program according to claim 62, wherein the program further causes the computer to execute
a step for performing the image processing on a skin image to be diagnosed and
a step for obtaining a diagnosis result for the skin image to be diagnosed using the predetermined system with the use of the parameter combination selected in the fourth step.

64. The diagnosis program according to claim 62, characterized in that the predetermined system is a neural network system.

65. The diagnosis program according to claim 62, characterized in that at least one of the parameter evaluations is performed by employing a leave-one-out method.

66. The diagnosis program according to claim 62, characterized in that the skin lesion which is a subject of the diagnosis is melanoma.

67. A storage storing the program defined in claim 62, in a machine-readable form.

68. A method for screening a cosmetic agent or a drug for diminishing a pigmentary deposition of skin using the remote diagnosis apparatus according to claim 52.

69. A method for screening a cosmetic agent or a drug for diminishing a pigmentary deposition of skin using the remote diagnosis system according to claim 57.

70. A method for screening a cosmetic agent or a drug for diminishing a pigmentary deposition of skin using the remote diagnosis method according to claim 61.
